(19) United States
(12) Patent Application Publication
(10) Pub. No.: US 2026/0335530 A1
(43) Pub. Date: Oct. 29, 2026

(54) CANISTER STATUS DETERMINATION FOR NEGATIVE PRESSURE WOUND THERAPY DEVICES

(71) Applicant: T.J.Smith and Nephew,Limited, Hull (GB)

(72) Inventors: Ben Alan Askem, Leeds (GB); David Joseph Brearley, Hessle (GB); Robert Gregory, East Yorkshire (GB); Allan Kenneth Frazer Grugeon Hunt, Beverley (GB); Daniel Stewart McKinlay, Hull (GB); Christopher James Milner, Hedon (GB); Felix Clarence Quintanar, Hull (GB); Kirsty Margaret Strachan, Hull (GB)

(21) Appl. No.: 19/116,241

(22) PCT Filed: Sep. 27, 2023
(86) PCT No.: PCT/EP2023/076771

§ 371 Date: Mar. 27, 2025

(30) Foreign Application Priority Data
Sep. 27, 2022 (GB) 2214047.9
Sep. 27, 2022 (GB) 2214052.9

(51) Int. Cl. A61M 1/00

(52) U.S. Cl. CPC A61M 1/96; A61M 1/982; A61M 2205/3317; A61M 2205/3379

(57) ABSTRACT

A negative pressure wound therapy device can include one or more fluid detection systems. A canister fluid level detection system can incorporate various fluid detection devices to communicate data relating to the fluid level of the canister. In some cases, a negative pressure wound therapy device can include a device housing, a negative pressure source, and a canister configured to be in fluid communication with the negative pressure source. The canister can include a canister housing configured to store fluid aspirated from a wound, a cap connected to the canister housing, and a fluid level sensor supported by the cap. The fluid level sensor can be configured to detect a completed electrical circuit when the fluid aspirated from the wound comes into contact with the sensor. An electronic circuitry can be configured to detect a state of the sensor and provide an indication of a status of the canister.

This application claims priority to U.K. Provisional Application No. 2214052.9, filed Sep. 27, 2022, titled “CANISTER STATUS DETERMINATION FOR NEGATIVE PRESSURE WOUND THERAPY DEVICES,” and U.K. Provisional Application No. 2214047.9, filed Sep. 27, 2022, titled “CANISTER STATUS DETERMINATION FOR NEGATIVE PRESSURE WOUND THERAPY DEVICES,” the entirety of each of which is hereby incorporated by reference and made part of this disclosure.

### TECHNICAL FIELD

Embodiments described herein relate to apparatuses, systems, and methods for the treatment of wounds, for example using dressings in combination with negative pressure wound therapy.

### DESCRIPTION OF THE RELATED ART

Many different types of wound dressings are known for aiding in the healing process of a human or animal. These different types of wound dressings include many different types of materials and layers, for example, gauze, pads, foam pads or multi-layer wound dressings. Topical negative pressure (TNP) therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds, and abdominal wounds or the like. TNP therapy assists in the closure and healing of wounds by reducing tissue edema, encouraging blood flow, stimulating the formation of granulation tissue, removing excess exudates and may reduce bacterial load. Thus, reducing infection to the wound. Furthermore, TNP therapy permits less outside disturbance of the wound and promotes more rapid healing.

### SUMMARY

In one aspect, a negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound; a cap connected to the canister housing and configured to be connected to the device housing when the canister is removably attached to the device housing; and a fluid level sensor supported by the cap, the fluid level sensor comprising a first and second arm extending into an interior of the canister housing and configured to be in fluid communication with fluid aspirated from the wound, wherein the fluid level sensor is configured to detect a completed electrical circuit and/or an electrical circuit state change when the fluid aspirated from the wound comes into contact with the first and second arm of the fluid level sensor, and wherein the fluid level sensor is configured to detect a canister full condition when the electrical circuit is completed and/or there is state change in the electrical circuit; and an electronic circuitry configured to detect a state of the fluid level sensor and provide an indication of a status of the canister; wherein the first and second arm comprise a length and the length of the first arm is different from the second arm or the first arm is positioned in a non-parallel arrangement from the second arm. In some cases, the aspirated fluid can include at least one of blood, a gel, a non-Newtonian fluid, a pseudo-solid, a solid, and/or a suspending fluid.

The negative pressure wound therapy device of any of the preceding paragraphs and/or any of the apparatuses, systems, or devices disclosed herein can include one or more of the following features. In some cases, the length of the first arm can be longer than a length of the second arm. In some cases, the first arm and second arm can have the same length. In some cases, the first arm and second arm can be parallel to each other. In some cases, the first arm and second arm can be angular to each other. In some cases, the fluid level sensor can comprise one or more sets of arms.

In another aspects, a negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound; a cap connected to the canister housing and configured to be connected to the device housing when the canister is removably attached to the device housing; and a fluid level sensor supported by the cap, the fluid level sensor comprising arms extending into an interior of the canister housing and configured to be in fluid communication with fluid aspirated from the wound, wherein the fluid level sensor is configured to detect a completed electrical circuit and/or an electrical circuit state change when the fluid aspirated from the wound comes into contact with the arms of the fluid level sensor, and wherein the fluid level sensor is configured to detect a canister full condition when the electrical circuit is completed and/or there is state change in the electrical circuit; and an electronic circuitry configured to detect a state of the fluid level sensor and provide an indication of a status of the canister; and a mechanical shield comprising an inner surface, an outer surface opposite the inner surface, and an interior portion, the mechanical shield positioned inside the canister, wherein the inner surface of the mechanical shield is facing the fluid level sensor and the mechanical shield is configured to protect the fluid level sensor from liquid within the canister.

The negative pressure wound therapy device of any of the preceding paragraphs and/or any of the apparatuses, systems, or devices disclosed herein can include one or more of the following features. In some cases, the mechanical shield can comprise a first opening along a top portion of the mechanical shield, and a second opening along a bottom portion of the mechanical shield. In some cases, the mechanical shield can surround the fluid level sensor. In some cases, the mechanical shield can comprise a conical shape that surrounds the fluid level sensor or the arms of the fluid level sensors. In some cases, the mechanical shield can comprise a truncated square pyramid shape that surrounds the fluid level sensor or the arms of the fluid level sensors. In some cases, the mechanical shield can comprise a truncated rectangular pyramid shape that surrounds the fluid level sensor or the arms of the fluid level sensors. In some cases, the mechanical shield can comprise one or more guards extending from at least one portion of the interior surface of the of the mechanical shield and positioned along at least one of the first opening and second opening of the mechanical shield, the one or more guards configured to reduce the possibility of liquid accessing the interior portion of the mechanical shield. In some cases, the one or more guards can form a fluid pathway from an exterior of the mechanical shield to the interior portion of the mechanical shield, thereby allowing fluid aspirated from the wound to access the interior portion of the mechanical shield as the fluid level within the canister reaches a threshold fluid level.

The negative pressure wound therapy device of any of the preceding paragraphs and/or any of the apparatuses, systems, or devices disclosed herein can include one or more of the following features. In some cases, the negative pressure wound therapy device can have more than one shield. Each shield can include one or more fluid level sensors. In some cases, a first mechanical shield can include more fluid level sensors than a second mechanical shield. In some cases the first and second mechanical shields include the same number of fluid level sensors.

In another aspects, a negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound; a cap connected to the canister housing and configured to be connected to the device housing when the canister is removably attached to the device housing; a first fluid level sensor and a second fluid level sensor, the first fluid level sensor configured to detect a first completed electrical circuit and/or a first electrical circuit state change when the fluid aspirated from the wound comes into contact with the first fluid level sensor and the second fluid level sensor configured to detect a second completed electrical circuit and/or a second electrical circuit state change when the fluid aspirated from the wound comes into contact with the second fluid level sensor; a first mechanical shield and a second mechanical shield each comprising a top opening and a bottom opening, wherein the first mechanical shield surrounds the first fluid level sensor, and the second mechanical shield surrounds the second fluid level sensor; and an electronic circuitry configured to detect a state of the first fluid level sensor and the second fluid level sensor and provide an indication of a status of the fluid in the canister.

The negative pressure wound therapy device of any of the preceding paragraphs and/or any of the apparatuses, systems, or devices disclosed herein can include one or more of the following features. In some cases, the first fluid level sensor and the second fluid level sensor can be positioned at different elevations relative to a base of the canister. In some cases, the first fluid level sensor and the second fluid level sensor can be positioned at the same elevation relative to a base of the canister and at different lateral positions. In some cases, the first mechanical shield and the second mechanical shield each can comprise an inner surface, an outer surface opposite the inner surface, and an interior portion, the first mechanical shield and the second mechanical shield positioned inside the canister, wherein the inner surfaces of the first mechanical shield and the second mechanical shield are facing the first fluid level sensor and the second fluid level sensor respectively, and the first mechanical shield and the second mechanical shield are configured to protect the first fluid level sensor and the second fluid level sensor respectively from liquid within the canister. In some cases, the inner surfaces of the first and second mechanical shields surround the fluid and second fluid level sensors respectively. In some cases, the top opening of the first shield can be smaller than the top opening of the second shield, and the bottom opening of the first shield can be smaller than the bottom opening of the second shield. The top opening of the second shield can receive a fluid output from the bottom opening of the first shield. In some cases, the top opening of the second shield can be smaller than the top opening of the first shield, and the bottom opening of the second shield can be smaller than the bottom opening of the first shield. In some cases, the negative pressure wound therapy can comprise a third fluid level sensor configured to detect a completed electrical circuit and/or there is state change in the electrical circuit when the fluid aspirated from the wound comes into contact with the third fluid level sensor; and a third mechanical shield comprising a top opening and a bottom opening, wherein the third mechanical shield surrounds the third fluid level sensor. In some cases, the top opening of the third shield can be smaller than the top opening of the first shield and the second shield, and the bottom opening of the third shield can be smaller than the bottom opening of the first shield and the second shield. In some cases, the top opening of the third shield can be larger than the top opening of the first shield and the second shield, and the bottom opening of the third shield can be larger than the bottom opening of the first shield and the second shield. The top opening of the third shield can receive a fluid output from the bottom opening of the second shield.

In another aspects, a canister for negative pressure wound therapy can comprise a canister housing configured to store fluid aspirated from the wound; a fluid level sensor configured to be in fluid communication with fluid aspirated from the wound, wherein the fluid level sensor is further configured to detect a completed electrical circuit and/or there is state change in the electrical circuit when the fluid aspirated from the wound comes into contact with the fluid level sensor; and a mechanical shield surrounding the fluid level sensor; and an electronic circuitry configured to detect a state of the fluid level sensor and provide an indication of a status of the canister.

The canister for negative pressure wound therapy of any of the preceding paragraphs and/or any of the apparatuses, systems, or devices disclosed herein can include one or more of the following features. In some cases, the fluid level sensor can comprise arms extending into an interior of the canister housing. In some cases, the mechanical shield can comprise an inner surface, an outer surface opposite the inner surface, and an interior portion, the mechanical shield positioned inside the canister, wherein the inner surface of the mechanical shield is facing the fluid level sensor and the mechanical shield is configured to protect the fluid level sensor from liquid within the canister. In some cases, the inner surface of the mechanical shield surrounds the fluid level sensor.

In one aspect, a negative pressure wound therapy device can comprise a device housing; a negative pressure source supported by the device housing, the negative pressure source configured to provide negative pressure to a wound covered by a wound dressing; a reader; and a canister configured to be in fluid communication with the negative pressure source and the wound dressing, the canister comprising: a canister housing configured to store fluid aspirated from the wound; and at least one physical marker configured to provide an indication to the reader of at least one characteristic of the canister when the canister is attached to the device housing.

The negative pressure wound therapy device of any of the preceding paragraphs and/or any of the apparatuses, systems, or devices disclosed herein can include one or more of the following features. In some cases, the at least one characteristic of the canister comprises at least one of a volume, size, and shape. In some cases, the at least one physical marker can include a shape and a size.

In another aspects, a negative pressure wound therapy device can comprise a device housing; a negative pressure source supported by the device housing, the negative pressure source configured to provide negative pressure to a wound covered by a wound dressing; a reader; and a canister configured to be in fluid communication with the negative pressure source and the wound dressing, the canister comprising: a canister housing configured to store fluid aspirated from the wound; and at least one metal configured to provide an indication to the reader of at least one characteristic of the canister when the canister is attached to the device housing.

The negative pressure wound therapy device of any of the preceding paragraphs and/or any of the apparatuses, systems, or devices disclosed herein can include one or more of the following features. In some cases, the reader can be configured to identify the at least one marker via at least one of electrical sensing shielding and near-field communication technology. In some cases, the at least one characteristic of the canister can comprise at least one of a volume, size, and shape. In some cases, the at least one metal marker can comprise a shape and a size and is positioned on a top portion of the canister. The at least one marker can include a conductive marker. In some cases, the at least one marker comprises a metal marker.

In another aspects, a negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising a canister housing configured to store fluid aspirated from the wound; and a sensor path at least partially extending from the wound dressing to the canister, the sensor path comprising: a first membrane; a second membrane; at least one spring positioned between the first and second membranes; a first conductive path positioned on the first membrane; and a second conductive path positioned on the second membrane; wherein in an open position, the first and second conductive paths are not in contact with each other; and wherein in a closed position, the first and second conductive paths are in contact with each other causing the first and second conductive paths to complete a circuit and/or cause a state change in the electrical circuit thereby providing an indication of a condition of the negative pressure wound therapy device.

The negative pressure wound therapy device of any of the preceding paragraphs and/or any of the apparatuses, systems, or devices disclosed herein can include one or more of the following features. In some cases, the open position can occur when negative pressure has not been applied, negative pressure has been paused, and/or negative pressure has not reached a threshold value. In some cases, the closed position can occur when negative pressure is applied, or the negative pressure reaches a threshold value. In some cases, the negative pressure source stops providing negative pressure when the first and second conductive paths complete the circuit and/or there is state change in the electrical circuit. The negative pressure source can change a level of negative pressure or a fluid flow rate when the first and second conductive paths complete the circuit and/or there is a state change in the electrical circuit. In some cases, the condition of the negative pressure wound therapy device is that a negative pressure threshold is reached. The condition of the negative pressure wound therapy device can be a presence of negative pressure at the wound dressing. In some cases, the sensor path further comprises a dual-core cable, and wherein the first and second membranes, the at least one spring, and the first and second conductive paths at least partially extend inside the dual-core cable. In some cases, the sensor path further comprises a co-axial cable, and wherein the first and second membranes, the at least one spring, and the first and second conductive paths at least partially extend inside the co-axial cable.

In another aspects, a negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound, and a fluid level sensor supported by the canister, the fluid level sensor comprising a first arm and a second arm extending into an interior of the canister housing and configured to be in fluid communication with fluid aspirated from the wound; wherein at least one of the first and second arms of the fluid level sensor comprises a coating, the coating configured to dissolve when the coating is in contact with the fluid aspirated from the wound for a threshold period of time.

The negative pressure wound therapy device of any of the preceding paragraphs and/or any of the apparatuses, systems, or devices disclosed herein can include one or more of the following features. In some cases, the coating comprises at least one of a Poly Vinyl Alcohol, a water based Poly Vinyl Alcohol, a Poly Vinyl Acetate, silicon oxides, and silicon nitrides material. In some cases, the negative pressure wound therapy device can include at least one layer covering the coating of the first and second arms, the at least one layer configured to at least partially drain fluid splashes and fluid droplets from the first and second arms.

In another aspects, a negative pressure wound therapy device can include a device housing and a negative pressure source supported by the device housing. The negative pressure source can be configured to provide negative pressure to a wound covered by a wound dressing. The negative pressure wound therapy device can include a canister configured to be in fluid communication with the negative pressure source and the wound dressing. The canister can include a canister housing configured to store fluid aspirated from the wound, and a fluid level sensor supported by the canister. In some cases, the fluid level sensor can include a first arm and a second arm extending into an interior of the canister housing. The first and second arms can be configured to be in fluid communication with fluid aspirated from the wound. In some cases, the first and second arms of the fluid level sensor can include a coating. A physical property of the coating can be configured to change when the coating is in contact with the fluid aspirated from the wound for a threshold period of time.

In another aspects, a negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound; and a fluid level sensor supported by the canister, the fluid level sensor comprising a first arm and a second arm extending into an interior of the canister housing and configured to be in fluid communication with fluid aspirated from the wound; a material positioned between the first and second arms of the fluid level sensor, the material comprising a dissolvable material, the dissolvable material configured to dissolve when the fluid aspirated from the wound comes into contact with the dissolvable material, wherein the dissolvable material is configured to change a signal of a circuit created by the dissolvable material and the first and second arms of the fluid level sensor when the dissolvable material dissolves, and wherein the fluid level sensor is configured to detect a canister full condition based on the change of the signal of the circuit created by the dissolvable material and the first and second arms of the fluid level sensor.

The negative pressure wound therapy device of any of the preceding paragraphs and/or any of the apparatuses, systems, or devices disclosed herein can include one or more of the following features. In some cases, the first and second arms of the fluid level sensor can include a semi-permeable membrane configured to prevent salts contained in the fluid aspirated from the wound from dissolving the dissolvable material but allowing liquids to move through the semi-permeable membrane and dissolve the dissolvable material. In some cases, the first and second arms of the fluid level sensor can be configured to spring towards each other when the dissolvable material dissolves.

In another aspects, a negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound; and a fluid level sensor supported by the canister, the fluid level sensor comprising a first arm and a second arm extending into an interior of the canister housing and configured to be in fluid communication with fluid aspirated from the wound; and a material positioned between the first and second arms of the fluid level sensor, the material comprising an expanding material, the expanding material configured to expand when the fluid aspirated from the wound comes into contact with the expanding material causing the first and second arms to move away from each other; wherein the first and second arms moving away from each other changes a signal of a circuit created by the first and second arms of the fluid level sensor; and wherein the fluid level sensor is configured to detect a canister full condition based on the change of the signal of the circuit created by first and second arms of the fluid level sensor.

The negative pressure wound therapy device of any of the preceding paragraphs and/or any of the apparatuses, systems, or devices disclosed herein can include one or more of the following features. In some cases, the first and second arms moving away from each other causes the circuit created by the first and second arms to break thereby changing the signal of the circuit.

In some aspects, a negative pressure wound therapy device can comprise a device housing and a negative pressure source supported by the device housing. The negative pressure source can be configured to provide negative pressure to a wound covered by a wound dressing. In some cases, the negative pressure wound therapy device can include a canister configured to be in fluid communication with the negative pressure source and the wound dressing. The canister can include a canister housing configured to store fluid aspirated from the wound, and a fluid level sensor supported by the canister. The fluid level sensor can include a first arm and a second arm extending into an interior of the canister housing and configured to be in fluid communication with fluid aspirated from the wound. The negative pressure wound therapy device can include a material positioned inside the canister. The material can include an expanding material, the expanding material configured to expand when the fluid aspirated from the wound comes into contact with the expanding material causing a distance between the first and second arms to change. The change in distance between the first and second arms can change a signal of a circuit created by the first and second arms of the fluid level sensor. In some cases, the fluid level sensor can be configured to detect a canister condition based on the change of the signal of the circuit created by first and second arms of the fluid level sensor.

The negative pressure wound therapy device of any of the preceding paragraphs and/or any of the apparatuses, systems, or devices disclosed herein can include one or more of the following features. In some cases, the expanding material can cause the first and second arms to move toward each other. The first and second arms moving toward each other can change the signal of the circuit created by the first and second arms of the fluid level sensor.

A negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound; a cap connected to the canister housing and configured to be connected to the device housing when the canister is removably attached to the device housing; and a fluid level sensor supported by the cap, the fluid level sensor comprising a first arm and a second arm extending into an interior of the canister housing and configured to be in fluid communication with fluid aspirated from the wound, wherein the fluid level sensor is configured to detect a level of fluid in the canister and/or a canister full condition responsive to the fluid aspirated from the wound coming into contact with the first and second arm of the fluid level sensor; and an electronic circuitry configured to detect a state of the fluid level sensor and provide an indication of a status of the canister; wherein the first and second arms comprise conductive, inductive, and/or capacitive path configured to facilitate detection of the level of fluid in the canister and/or a canister full condition.

The negative pressure wound therapy device of any of the preceding paragraphs and/or any of the apparatuses, systems, or devices disclosed herein can include one or more of the following features. The first and second arms can comprise a shape that changes along a length of the first and second arm. The first arm and second arm can have the same length. The first arm and second arm can be parallel to each other. The first arm and second arm can be angular to each other. The states of the fluid level sensor can comprise a multi-bit value indicative of a level of fluid in the canister.

A negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound; and at least one sensor supported by the canister housing, wherein the at least one sensor is configured to determine viscosity of the fluid or a change of viscosity of the fluid entering the canister or within the canister; and an electronic circuitry configured to detect a state of the at least one sensor and provide an indication of the viscosity of the fluid or a change of viscosity of the fluid.

The negative pressure wound therapy device of any of the preceding paragraphs and/or any of the apparatuses, systems, or devices disclosed herein can include one or more of the following features. The electronic circuitry can be configured to detect a first unique identifier based on determining how the at least one sensor is covered and/or uncovered by liquid. The at least one sensor can comprise a first sensor and a second sensor, wherein the first sensor is at a first position in a fluid flow path and the second sensor is at a second position in the fluid flow path, wherein the second position is downstream of the first position; wherein the electronic circuitry is configured to detect a unique identifier based on determining how the first and second sensors are covered and/or uncovered by fluid in the canister, and wherein the unique identifier is used to determine a viscosity measurement and/or a change in viscosity of the fluid aspirated from the wound. The at least one sensor can be configured to determine viscosity of the fluid within the canister based on a period of coverage of the sensor during movement of the canister. Coverage of the sensor can be determined based on detecting a level of fluid in the canister using the at least one sensor. The negative pressure wound therapy device can further comprise an inertial measurement unit (IMU) configured to be used for calibration of the at least one sensor.

A negative pressure wound therapy device can comprise a device housing; a negative pressure source supported by the device housing, the negative pressure source configured to provide negative pressure to a wound covered by a wound dressing; a canister configured to be in fluid communication with the negative pressure source and the wound dressing, the canister comprising: a canister housing configured to store fluid aspirated from the wound; a viscosity measurement device positioned within an interior of the canister and configured to be connected to the device housing when the canister is removably attached to the device housing.

The negative pressure wound therapy device of any of the preceding paragraphs and/or any of the apparatuses, systems, or devices disclosed herein can include one or more of the following features. The viscosity measurement device can comprise a rotational viscometer including: a paddle configured to be positioned within the interior of the canister, wherein the rotational viscometer is configured to measure viscosity based on calculating a frictional force on the paddle caused by the fluid due to a level of extra energy required to maintain a spinning speed of the paddle. The viscosity measurement device can comprise a vibro-viscometer including: a paddle configured to be positioned within the interior of the canister, wherein the vibro-viscometer is configured to measure viscosity based on a driving current required to maintain a vibrational frequency through the fluid in the canister. The paddle can be configured to be positioned within a test chamber within the interior of the canister.

In some aspects, a negative pressure wound therapy device can comprise a device housing and a negative pressure source supported by the device housing. The negative pressure source can be configured to provide negative pressure to a wound covered by a wound dressing. The negative pressure wound therapy device can include a canister configured to be in fluid communication with the negative pressure source and the wound dressing. The canister can include a canister housing configured to store aspirated fluid, a cap connected to the canister housing and configured to be connected to the device housing when the canister is removably attached to the device housing; and a fluid level sensor configured to detect a level of fluid in the canister and/or a canister full condition. The negative pressure wound therapy device can include an electronic circuitry configured to detect a state of the fluid level sensor and provide an indication of a status of the canister.

The negative pressure wound therapy device of any of the preceding paragraphs and/or any of the apparatuses, systems, or devices disclosed herein can include one or more of the following features. The fluid level sensor can be configured to detect the level of fluid in the canister and/or the canister full condition responsive to the aspirated fluid contacting the fluid level sensor. The fluid level sensor can include a conductive sensor configured to facilitate detection of the level of fluid in the canister and/or a canister full condition. The fluid level sensor can include an inductive sensor configured to facilitate detection of the level of fluid in the canister and/or a canister full condition. The fluid level sensor can include a capacitive sensor configured to facilitate detection of the level of fluid in the canister and/or a canister full condition. The fluid level sensor can include at least one arm or path. In some cases, the fluid level sensor can include a first arm and a second arm. In some cases, the aspirated fluid can include at least one of blood, a gel, a non-Newtonian fluid, a pseudo-solid, a solid, and a suspending fluid. The fluid level sensor can be supported by the cap and extend into an interior of the canister.

In some aspects, a negative pressure wound therapy device can comprise a device housing and a negative pressure source supported by the device housing. The negative pressure source can be configured to provide negative pressure to a wound covered by a wound dressing. The negative pressure wound therapy device can include a canister configured to be in fluid communication with the negative pressure source and the wound dressing. The canister can include a canister housing configured to store fluid aspirated from the wound, and at least one sensor configured to determine viscosity of the fluid or a change of viscosity of the fluid entering the canister or within the canister. The negative pressure wound therapy device can include an electronic circuitry configured to detect a state of the at least one sensor and provide an indication of the viscosity of the fluid or a change of viscosity of the fluid.

The negative pressure wound therapy device of any of the preceding paragraphs and/or any of the apparatuses, systems, or devices disclosed herein can include one or more of the following features. The at least one sensor can be supported by the device housing. In some cases, the at least one sensor can be supported by the canister housing. The negative pressure wound therapy device can include a cap connected to the canister housing and configured to be connected to the device housing when the canister is removably attached to the device housing. The at least one sensor can be supported by the canister housing.

Disclosed herein are methods of operating a negative pressure wound therapy device of any of the preceding paragraphs and/or any of the devices, apparatuses, or systems disclosed herein.

Disclosed herein are kits that include the negative pressure wound therapy device of any of the preceding paragraphs and/or any of the devices, apparatuses, or systems disclosed herein and one or more wound dressings.

Any of the features, components, or details of any of the arrangements or embodiments disclosed in this application, including without limitation any of the apparatus embodiments and any of the negative pressure wound therapy embodiments disclosed herein, are interchangeably combinable with any other features, components, or details of any of the arrangements or embodiments disclosed herein to form new arrangements and embodiments.

### BRIEF DESCRIPTION OF THE DRAWINGS

FIG. 1A illustrates a negative pressure wound therapy system.

FIG. 1B illustrates another negative pressure wound therapy system.

FIG. 2A is an isometric view of a negative pressure wound therapy device and canister, showing the canister detached from the pump assembly of the device.

FIG. 2B is a back view of the negative pressure wound therapy device shown in FIG. 2A.

FIG. 2C illustrates a top surface of the negative pressure wound therapy device shown in FIG. 2A, showing a graphical user interface.

FIG. 3 illustrates a schematic of a control system of a negative pressure wound therapy device.

FIG. 4 illustrates another negative pressure wound therapy system.

FIG. 5A illustrates an exploded view of a canister top and associated components.

FIG. 5B-5E illustrates exploded views of a canister top and the pump assembly components.

FIGS. 6A-6D illustrate a canister status detection system for a negative pressure wound therapy system.

FIG. 6E illustrates multiple views of a fluid level sensor.

FIG. 7-9 illustrate examples of a fluid level sensor.

FIG. 10 illustrates an example of a fluid level sensor.

FIG. 11 illustrates a graph of resistance, inductance, and capacitance over time.

FIG. 12 illustrates a cross-section view of a fluid level sensor and a mechanical shield.

FIGS. 13, 15, and 17 illustrate examples of a mechanical shield.

FIGS. 14, 16, 18 and 19 illustrate cross-section views of the examples of a mechanical shield shown in FIGS. 13, 15, and 17.

FIGS. 20, 28-29, 32-33 illustrate cross-section views of examples of a mechanical shield.

FIGS. 21 and 22 illustrate examples of a canister cap including windows defining a mechanical shield.

FIGS. 23A-23B illustrate a cross-section view of an example of a canister cap including one or more windows.

FIG. 24 illustrates an example of a portion of a cap assembly including a window defining a mechanical shield.

FIG. 25A illustrates an example of a canister cap including one or more windows defining a mechanical shield.

FIG. 25B illustrates a cross-section view of the canister cap shown in in FIG. 25A.

FIG. 26 illustrates a cross-section view of a canister including a canister cap assembly.

FIGS. 27A-27C illustrate different views of an example of a canister cap assembly including one or more windows defining a mechanical shield.

FIGS. 30A-30B illustrate a cross-section view of a canister in a stable position.

FIG. 30C illustrates a cross-section view of a canister in an unstable position.

FIGS. 31A and 31B illustrate examples of an NFC reader and an NFC tag antenna.

FIG. 34 illustrates a cross-section view of an example of a mechanical shield and multiple fluid level sensors.

FIG. 35 illustrates a top section view of the mechanical shield of FIG. 34.

FIGS. 36A-36B illustrate an example of a fluid level sensor.

FIGS. 37A-38 illustrate an example of a negative pressure wound therapy device including a sensor path extending from a wound dressing to a canister.

FIGS. 39A-39B illustrate an example of a canister body including an integral cap assembly.

FIG. 40 illustrates an example of a rotational viscometer.

FIG. 41 illustrates an example of a rotational viscometer within a canister and pump system.

FIG. 42 illustrates an example of a vibro-viscometer.

### DETAILED DESCRIPTION

Embodiments disclosed herein relate to systems and methods of treating and/or monitoring a wound. Some embodiments of the negative pressure wound therapy devices disclosed herein can include a negative pressure source configured to be connected and/or fluidically coupled, via a fluid flow path, to a wound covered by a wound dressing and provide negative pressure to a wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a patient or otherwise that benefit from pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

Embodiments of systems and methods disclosed herein can be used with topical negative pressure (“TNP”) or reduced pressure therapy systems. Briefly, negative pressure wound therapy assists in the closure and healing of many forms of “hard to heal” wounds by reducing tissue oedema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. TNP therapy can help to stabilize the tissue in the apposed position of closure. A further beneficial use of TNP therapy can be found in grafts and flaps where removal of excess fluid is important and close proximity of the graft to tissue is required in order to ensure tissue viability.

As used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760-X) mmHg. In addition, negative pressure that is “less” or “smaller” than X mmHg corresponds to pressure that is closer to atmospheric pressure (for example, −40 mmHg is less than −60 mmHg). Negative pressure that is “more” or “greater” than-X mmHg corresponds to pressure that is further from atmospheric pressure (for example, −80 mmHg is more than −60 mmHg). In some cases, local ambient atmospheric pressure is used as a reference point, and such local atmospheric pressure may not necessarily be, for example, 760 mmHg.

Systems and methods disclosed herein can be used with other types of treatment in addition to or instead of reduced pressure therapy, such as irrigation, ultrasound, heat or cold, neuro stimulation, or the like. In some cases, disclosed systems and methods can be used for wound monitoring without application of additional therapy. Systems and methods disclosed herein can be used in conjunction with a dressing, including with compression dressing, reduced pressure dressing, or the like.

A healthcare provider, such as a clinician, nurse, or the like, can provide a TNP prescription specifying, for example, the pressure level or time of application. However, the healing process is different for each patient and the prescription may affect the healing process in a way the clinician or healthcare provider did not expect at the time of devising the prescription. A healthcare provider may try to adjust the prescription as the wound heals (or does not heal), but such process may require various appointments that can be time consuming and repetitive. Embodiments disclosed herein provide systems, devices, or methods of efficiently adjusting TNP prescriptions and delivering effective TNP therapy.

### Wound Therapy System

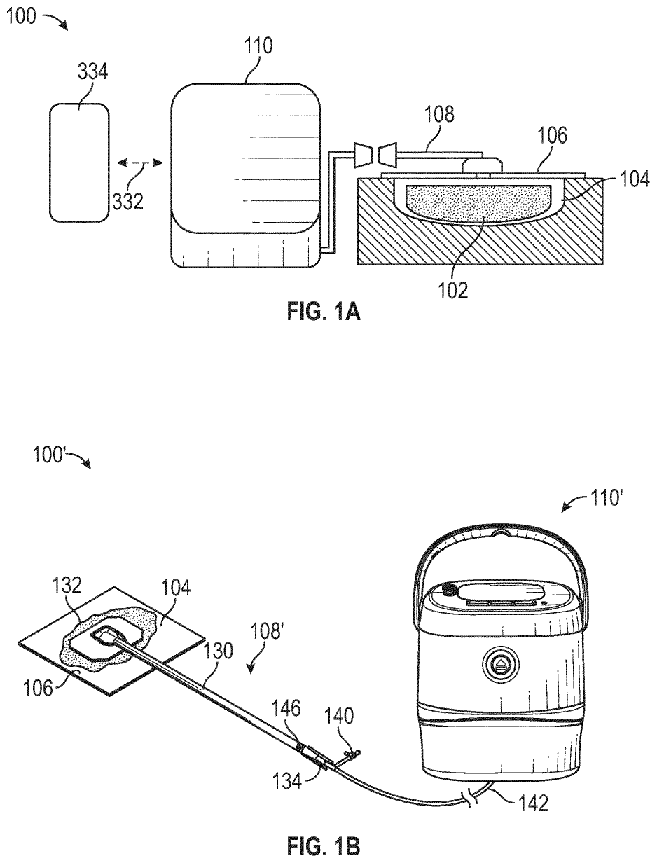

FIG. 1A schematically illustrates a negative pressure wound treatment system 100 (sometimes referred to as a reduced or negative pressure wound therapy system, a TNP system, or a wound treatment system). In any implementations disclosed herein, though not required, the negative pressure wound treatment system 100 can include a wound filler 102 placed on or inside a wound 104 (which may be a cavity). The wound 104 can be sealed by a wound cover 106, which can be a drape, such that the wound cover 106 can be in fluidic communication with the wound 104. The wound filler 102 in combination with the wound cover 106 can be referred to as a wound dressing. A tube or conduit 108 (also referred to herein as a flexible suction adapter or a fluidic connector) can be used to connect the wound cover 106 with a wound therapy device 110 (sometimes as a whole or partially referred to as a “pump assembly”) configured to supply reduced or negative pressure. The conduit 108 can be a single or multi lumen tube. A connector can be used to removably and selectively couple a conduit or tube of the device 110 with the conduit 108.

In any of the systems disclosed herein, a wound therapy device can be canisterless, wherein, for example and without limitation, wound exudate is collected in the wound dressing or is transferred via a conduit for collection at another location. However, any of the wound therapy devices disclosed herein can include or support a canister.

Additionally, with any of the wound therapy systems disclosed herein, any of the wound therapy devices can be mounted to or supported by the wound dressing or adjacent to the wound dressing. The wound filler 102 can be any suitable type, such as hydrophilic or hydrophobic foam, gauze, inflatable bag, and so on. The wound filler 102 can be conformable to the wound 104 such that the wound filler 102 substantially fills the cavity of the wound 104. The wound cover 106 can provide a substantially fluid impermeable seal over the wound 104. The wound cover 106 can have a top side and a bottom side. The bottom side can adhesively (or in any other suitable manner) seal with the wound 104, for example by sealing with the skin around the wound 104. The conduit 108 or any other conduit disclosed herein can be formed from polyurethane, PVC, nylon, polyethylene, silicone, or any other suitable material.

The wound cover 106 can have a port (not shown) configured to receive an end of the conduit 108. In some cases, the conduit 108 can otherwise pass through or under the wound cover 106 to supply reduced pressure to the wound 104 so as to maintain a desired level of reduced pressure in the wound 104. The conduit 108 can be any suitable article configured to provide at least a substantially sealed fluid flow pathway or path between the wound therapy device 110 and the wound cover 106, so as to supply the reduced pressure provided by the wound therapy device 110 to wound 104.

The wound cover 106 and the wound filler 102 can be provided as a single article or an integrated single unit. In some cases, no wound filler is provided and the wound cover by itself may be considered the wound dressing. The wound dressing can then be connected, via the conduit 108, to a source of negative pressure of the wound therapy device 110. In some cases, though not required, the wound therapy device 110 can be miniaturized and portable, although larger conventional negative pressure sources (or pumps) can also be used.

The wound cover 106 can be located over a wound site to be treated. The wound cover 106 can form a substantially sealed cavity or enclosure over the wound. The wound cover 106 can have a film having a high water vapour permeability to enable the evaporation of surplus fluid, and can have a superabsorbing material contained therein to safely absorb wound exudate. In some cases, the components of the TNP systems described herein can be particularly suited for incisional wounds that exude a small amount of wound exudate.

The wound therapy device 110 can operate with or without the use of an exudate canister. In some cases, as is illustrated, the wound therapy device 110 can include an exudate canister. In some cases, configuring the wound therapy device 110 and conduit 108 so that the conduit 108 can be quickly and easily removed from the wound therapy device 110 can facilitate or improve the process of wound dressing or pump changes, if necessary. Any of the pump assemblies disclosed herein can have any suitable connection between the conduit 108 and the pump.

The wound therapy device 110 can deliver negative pressure of approximately −80 mmHg, or between about −20 mmHg and −200 mmHg. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 110.

As will be described in greater detail below, the negative pressure wound treatment system 100 can be configured to provide a connection 332 to a separate or remote computing device 334. The connection 332 can be wired or wireless (such as, Bluetooth, Bluetooth low energy (BLE), Near-Field Communication (NFC), WiFi, or cellular). The remote computing device 334 can be a smartphone, a tablet, a laptop or another standalone computer, a server (such as, a cloud server), another pump device, or the like.

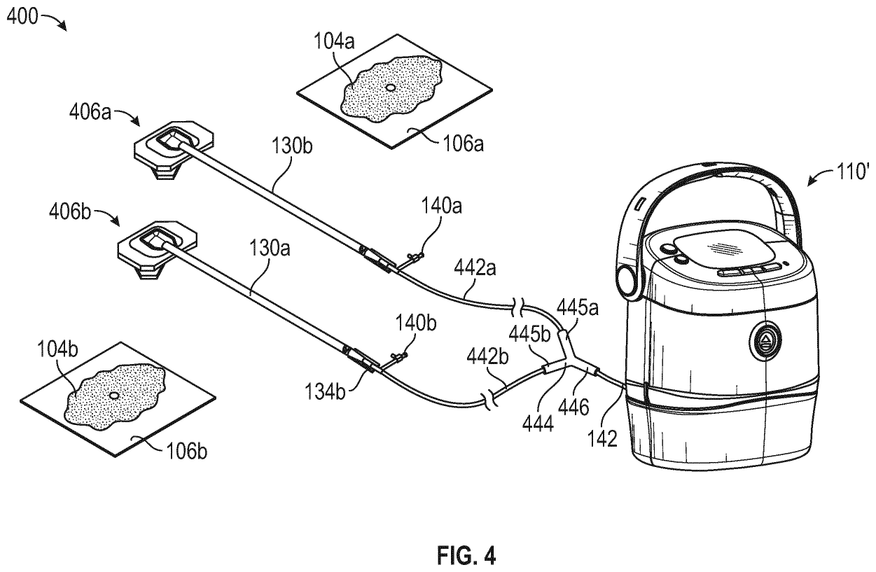

FIG. 1B illustrates another negative pressure wound treatment system 100′. The negative pressure wound treatment system 100′ can have any of the components, features, or other details of any of the other negative pressure wound treatment system disclosed herein, including without limitation the negative pressure wound treatment system 100 illustrated in FIG. 1A or the negative pressure wound treatment system 400 illustrated in FIG. 4, in combination with or in place of any of the components, features, or other details of the negative pressure wound treatment system 100′ shown in FIG. 1B and/or described herein. The negative pressure wound treatment system 100′ can have a wound cover 106 over a wound 104 that can seal the wound 104. A conduit 108′, such as a single or multi lumen tube can be used to connect the wound cover 106 with a wound therapy device 110′ (sometimes as a whole or partially referred to as a “pump assembly”) configured to supply reduced or negative pressure. The wound cover 106 can be in fluidic communication with the wound 104.

With reference to FIG. 1B, the conduit 108′ can have a bridge portion 130 that can have a proximal end portion and a distal end portion (the distal end portion being closer to the wound 104 than the proximal end portion, and an applicator 132 at the distal end of the bridge portion 130 forming the flexible suction adapter (or conduit) 108′. A connector 134 can be disposed at the proximal end of the bridge portion 130, so as to connect to at least one of the channels that can extend along a length of the bridge portion 130 of the conduit 108 shown in FIG. 1B. A cap 140 can be coupled with a portion of the conduit 108 and can, in some cases, as illustrated, be attached to the connector 134. The cap 140 can be useful in preventing fluids from leaking out of the proximal end of the bridge portion 130. The conduit 108′ can be a Soft Port manufactured by Smith & Nephew. As mentioned, the negative pressure wound treatment system 100′ can include a source of negative pressure, such as the device 110′, capable of supplying negative pressure to the wound 104 through the conduit 108′. Though not required, the device 110′ can also include a canister or other container for the storage of wound exudates and other fluids that can be removed from the wound.

The device 110′ can be connected to the connector 134 via a conduit or tube 142. In use, the applicator 132 can be placed over an aperture formed in a cover 106 that is placed over a suitably-prepared wound or wound 104. Subsequently, with the wound therapy device 110′ connected via the tube 142 to the connector 134, the wound therapy device 110′ can be activated to supply negative pressure to the wound. Application of negative pressure can be applied until a desired level of healing of the wound is achieved.

The bridge portion 130 can comprise an upper channel material or layer positioned between an upper layer and an intermediate layer, with a lower channel material or layer positioned between the intermediate layer and a bottom layer. The upper, intermediate, and lower layers can have elongate portions extending between proximal and distal ends and can include a material that is fluid-impermeable, for example polymers such as polyurethane. It will of course be appreciated that the upper, intermediate, and lower layers can each be constructed from different materials, including semi-permeable materials. In some cases, one or more of the upper, intermediate, and lower layers can be at least partially transparent. In some instances, the upper and lower layers can be curved, rounded or outwardly convex over a majority of their lengths.

The upper and lower channel layers can be elongate layers extending from the proximal end to the distal end of the bridge 130 and can each comprise a porous material, including for example open-celled foams such as polyethylene or polyurethane. In some cases, one or more of the upper and lower channel layers can be comprised of a fabric, for example a knitted or woven spacer fabric (such as a knitted polyester 3D fabric, Baltex 7970®, or Gehring 879®) or a nonwoven material, or terry-woven or loop-pile materials. The fibers may not necessarily be woven, and can include felted and flocked (including materials such as Flotex®) fibrous materials. The materials selected are preferably suited to channeling wound exudate away from the wound and for transmitting negative pressure or vented air to the wound site, and can also confer a degree of kinking or occlusion resistance to the channel layers. In one example, the upper channel layer can include an open-celled foam such as polyurethane, and the lower channel layer can include a fabric. In another example, the upper channel layer is optional, and the system can instead be provided with an open upper channel. The upper channel layer can have a curved, rounded or upwardly convex upper surface and a substantially flat lower surface, and the lower channel layer can have a curved, rounded or downwardly convex lower surface and a substantially flat upper surface.

The fabric or material of any components of the bridge 130 can have a three-dimensional (3D) structure, where one or more types of fibers form a structure where the fibers extend in all three dimensions. Such a fabric can in some cases aid in wicking, transporting fluid or transmitting negative pressure. In some cases, the fabric or materials of the channels can include several layers of material stacked or layered over each other, which can in some cases be useful in preventing the channel from collapsing under the application of negative pressure. The materials used in some implementations of the conduit 108′ can be conformable and pliable, which can, in some cases, help to avoid pressure ulcers and other complications which can result from a wound treatment system being pressed against the skin of a patient.

The distal ends of the upper, intermediate, and lower layers and the channel layers can be enlarged at their distal ends (to be placed over a wound site), and can form a “teardrop” or other enlarged shape. The distal ends of at least the upper, intermediate, and lower layers and the channel layers can also be provided with at least one through aperture. This aperture can be useful not only for the drainage of wound exudate and for applying negative pressure to the wound, but also during manufacturing of the device, as these apertures can be used to align these respective layers appropriately.

In some implementations, a controlled gas leak 146 (sometimes referred to as gas leak, air leak, or controlled air leak) can be disposed on the bridge portion 130, for example at the proximal end thereof. This air leak 146 can comprise an opening or channel extending through the upper layer of the bridge portion 130, such that the air leak 146 is in fluidic communication with the upper channel of the bridge portion 130. Upon the application of suction to the conduit 108, gas (such, as air) can enter through the gas leak 146 and move from the proximal end of the bridge portion 130 to the distal end of the bridge portion along the upper channel of the bridge portion 130. The gas can then be suctioned into the lower channel of the bridge portion 130 by passing through the apertures through the distal ends of the upper, intermediate, and lower layers.

The air leak 146 can include a filter. The air leak 146 can be located at the proximal end of the bridge portion 130 so as to minimize the likelihood of wound exudate or other fluids coming into contact and possibly occluding or interfering with the air leak 146 or the filter. In some instances, the filter can be a microporous membrane capable of excluding microorganisms and bacteria, and which may be able to filter out particles larger than 45 μm. Preferably, the filter can exclude particles larger than 1.0 μm, and more preferably, particles larger than 0.2 μm. Advantageously, some implementations can provide for a filter that is at least partially chemically-resistant, for example to water, common household liquids such as shampoos, and other surfactants. In some cases, reapplication of vacuum to the suction adapter or wiping of the exposed outer portion of the filter may be sufficient to clear any foreign substance occluding the filter. The filter can be composed of a suitably-resistant polymer such as acrylic, polyethersulfone, or polytetrafluoroethylene, and can be oleophobic or hydrophobic. In some cases, the gas leak 146 can supply a relatively constant gas flow that does not appreciably increase as additional negative pressure is applied to the conduit 108′. In instances of the negative pressure wound treatment system 100 where the gas flow through the gas leak 146 increases as additional negative pressure is applied, preferably this increased gas flow will be minimized and not increase in proportion to the negative pressure applied thereto. Further description of such bridges, conduits, air leaks, and other components, features, and details that can be used with any implementations of the negative pressure wound treatment systems disclosed herein are found in U.S. Pat. No. 8,801,685, which is incorporated by reference in its entirety as if fully set forth herein.

Any of the wound therapy devices (such as, the device 110 or 110′) disclosed herein can provide continuous or intermittent negative pressure therapy. Continuous therapy can be delivered at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. Intermittent therapy can be delivered between low and high negative pressure set points (sometimes referred to as setpoint). Low set point can be set at above 0 mmHg, −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, or below −180 mmHg. High set point can be set at above −25 mmHg, −40 mmHg, −50 mmHg, −60 mmHg, −70 mmHg, −80 mmHg, −90 mmHg, −100 mmHg, −120 mmHg, −125 mmHg, −140 mmHg, −160 mmHg, −180 mmHg, −200 mmHg, or below −200 mmHg. During intermittent therapy, negative pressure at low set point can be delivered for a first time duration, and upon expiration of the first time duration, negative pressure at high set point can be delivered for a second time duration. Upon expiration of the second time duration, negative pressure at low set point can be delivered. The first and second time durations can be same or different values. In some cases, the wound therapy devices disclosed herein can provide negative pressure to a portion of the wound dressing only. Providing negative pressure to a portion of the wound dressing can allow negative pressure wound therapy to be applied to only a portion of the wound area covered by the wound dressing. This can beneficially allow clinicians to apply therapy to a specific portion or portions of a wound if needed.

In operation, the wound filler 102 can be inserted into the cavity of the wound 104, and wound cover 106 can be placed so as to seal the wound 104. The wound therapy device 110′ can provide negative pressure to the wound cover 106, which can be transmitted to the wound 104 via the wound filler 102. Fluid (such as, wound exudate) can be drawn through the conduit 108′ and stored in a canister. In some cases, fluid is absorbed by the wound filler 102 or one or more absorbent layers (not shown).

Wound dressings that can be utilized with the pump assembly and systems of the present application include Renasys-F, Renasys-G, Renasys AB, and Pico Dressings available from Smith & Nephew. Further description of such wound dressings and other components of a negative pressure wound therapy system that can be used with the pump assembly and systems of the present application are found in U.S. Patent Publication Nos. 2012/0116334, 2011/0213287, 2011/0282309, 2012/0136325, U.S. Pat. No. 9,084,845, and International App. No. PCT/EP2020/078376, each of which is incorporated by reference in its entirety as if fully set forth herein. In some cases, other suitable wound dressings can be utilized.

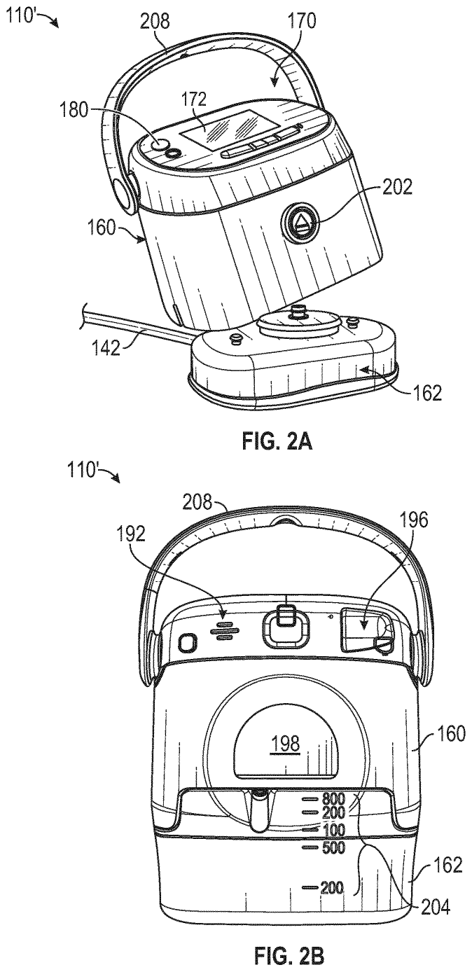

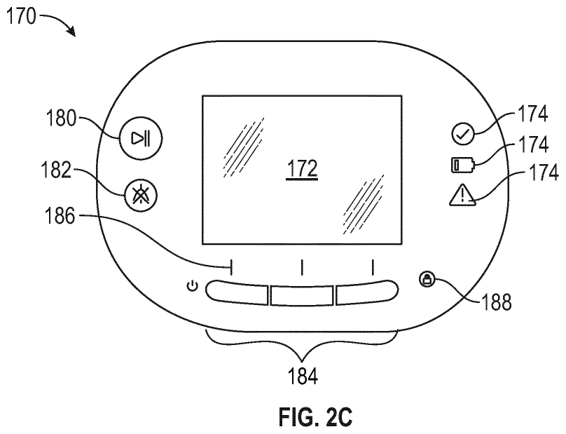

FIGS. 2A-2C show the negative pressure wound therapy device 110′. As illustrated, a pump assembly 160 and canister 162 can be connected, thereby forming the wound therapy device 110′. With reference to FIG. 2C, the pump assembly 160 can include an interface panel 170 having a display 172, one or more indicators 174, or one or more controls or buttons, including, for example and without limitation, a therapy start and pause button 180 or an alarm/alert mute button 182. The interface panel 170 can have one or more input controls or buttons 184 (three being shown) that can be used to control any functions of the pump assembly 160 or the interface panel 170. For example and without limitation, one or more of the buttons 184 can be used to turn the pump assembly 160 on or off, to start or pause therapy, to operate and monitor the operation of the pump assembly 160, to scroll through menus displayed on the display 172, or to control or perform other functions. In some cases, the command buttons 184 can be programmable, and can be made from a tactile, soft rubber.

Additionally, the interface panel 170 can have visual indicators 186 that can indicate which of the one or more buttons 184 is active. The interface panel 170 can also have a lock/unlock control or button 188 that can be configured to selectively lock or unlock the functionality of the various buttons (e.g., buttons 184) or the display 172. For example, therapy setting adjustment can be locked/unlocked via the lock/unlock control 188. When the lock/unlock button 188 is in the locked state, depressing one or more of the various other buttons or the display will not cause the pump assembly 160 to change any display functions or performance functions of the device. In some cases, when the lock/unlock button 188 is in the locked state, some buttons or portions of the display may be available and able to cause the pump assembly 160 to change any display functions or performance functions of the device while other buttons or portion of the display are not able to cause the pump assembly 160 to change any display functions or performance functions of the device. For example and without limitation, when the lock/unlock button 188 is in the locked state, the menu navigation may still be available and able to be used or activated, but the commands that adjust therapy settings are greyed out, and not able to be used or not able to cause a change of function of the device. This way, the interface panel 170 will be protected from inadvertent bumping or touching of the various buttons or display. The interface panel 170 can be located on an upper portion of the pump assembly 160, for example and without limitation on an upward facing surface of the pump assembly 160.

The display 172, which can be a screen such as an LCD screen, can be mounted in a middle portion of the interface panel 170. The display 172 can be a touch screen display. The display 172 can support playback of audiovisual (AV) content, such as instructional videos, and render a number of screens or graphical user interfaces (GUIs) for configuring, controlling, and monitoring the operation of the pump assembly 160.

The one or more indicators 174 can be lights (such as, LEDs) and can be configured to provide a visual indication of alarm conditions and/or a status of the pump. For example and without limitation, the one or more indicators 174 can be configured to provide a visual indication of a status of the pump assembly 160 or other components of the negative pressure wound treatment system 100′, including without limitation the conduit 108′ or the wound cover 106 (such as, to provide an indication of normal operation, low battery, a leak, canister full, blockage, overpressure, or the like). Any one or more suitable indicators can be additionally or alternatively used, such as visual, audio, tactile indicator, and so on.

FIG. 2B shows a back or rear view of the wound therapy device 110′ shown in the FIG. 2A. As shown, the pump assembly 160 can include a speaker 192 for producing sound. For example and without limitation, the speaker 192 can generate an acoustic alarm in response to deviations in therapy delivery, non-compliance with therapy delivery, or any other similar or suitable conditions or combinations thereof. The speaker 192 can provide audio to accompany one or more instructional videos that can be displayed on the display 172.

The pump assembly 160 can be configured to provide easy access (such as, an access door on the casing of the pump assembly) to one or more filters of the pump assembly 160, such as antibacterial filters. This can enable a user (such as, a healthcare provider or patient) to more easily access, inspect or replace such filters. The pump assembly 160 can also include a power jack 196 for providing power to the pump assembly 160 or for charging and recharging an internal power source (such as, a battery). Some implementations of the pump assembly 160 can include a disposable or renewable power source, such as one or more batteries, so that no power jack is needed. The pump assembly 160 can have a recess 198 formed therein to facilitate gripping of the pump assembly 160.

The canister 162 can hold fluid aspirated from the wound 104. In some cases, the aspirated fluid can include at least one of blood, a gel, a non-Newtonian fluid, a pseudo-solid, a solid, a suspending fluid, and/or a combination thereof. For example, the canister 162 can have an 800 mL (or approximately 800 mL) capacity, or from a 300 mL or less capacity to a 1000 mL or more capacity, or any capacity level in this range. The canister 162 can include a tubing for connecting to the conduit 108′ in order to form a fluid flow path. The canister 162 can be replaced with another canister, such as when the canister 162 has been filled with fluid. With reference to FIG. 2A, the wound therapy device 110′ can include a canister inlet tube 200 (also referred to herein as a dressing port connector) in fluid communication with the canister 162. For example and without limitation, the canister inlet tube 200 can be used to connect with the conduit 108′.

The canister 162 can be selectively coupleable and removable from the pump assembly 160. With reference to FIG. 2A, in some cases, a canister release button 202 can be configured to selectively release the canister 162 from the pump assembly 160. With reference to FIG. 2B, the canister 162 can have one or more fill lines or graduations 204 to indicate to the user and amount of fluid or exudate stored within the canister 162.

The wound therapy device 110′ can have a handle 208 that can be used to lift or carry the wound therapy device 110′. The handle 208 can be coupled with the pump assembly 160 and can be rotatable relative to the wound therapy device 110′ so that the handle can be rotated upward for lifting or carrying the wound therapy device 110′ or the pump assembly 160, or rotated into a lower profile in a more compact position when the handle is not being used. In some cases, the handle 208 can be coupled with the pump assembly 160 in a fixed position. The handle 208 can be coupled with an upper portion of the pump assembly 160 or can be removable from the wound therapy device 110′.

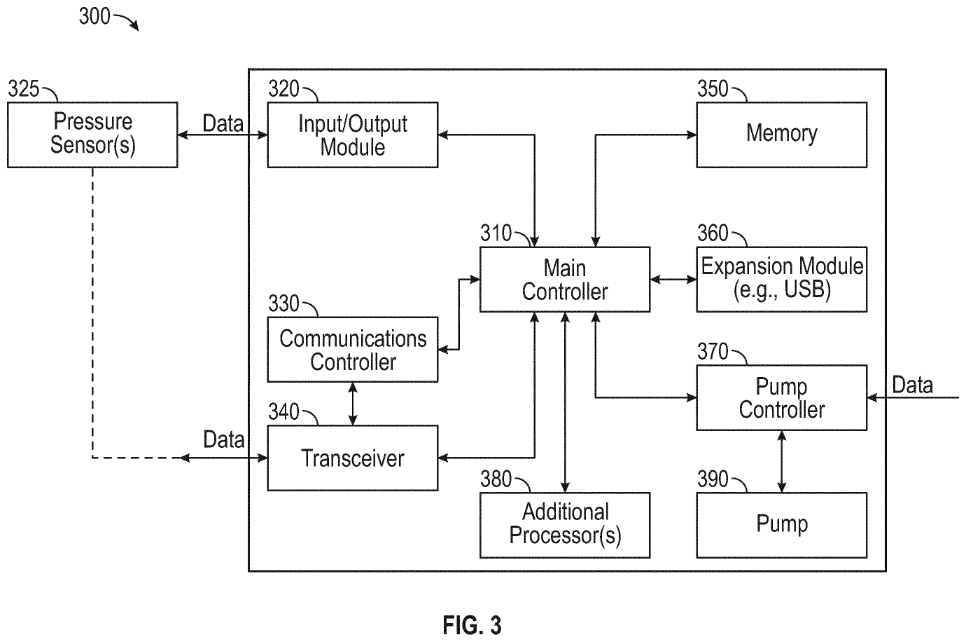

FIG. 3 illustrates a schematic of a control system 300 that can be employed in any of the wound therapy devices described herein, such as in the wound therapy device 110′. Electrical components can operate to accept user input, provide output to the user, operate the pressure source, provide connectivity, and so on. A first processor (such as, a main controller 310) can be responsible for user activity, and a second processor (such as, a pump controller 370) can be responsible for controlling another device, such as a pump 390.

An input/output (I/O) module 320 can be used to control an input and/or output to another component or device, such as the pump 390, one or more sensors (for example, one or more pressure sensors 325 configured to monitor pressure in one or more locations of the fluid flow path), or the like. For example, the I/O module can receive data from one or more sensors through one or more ports, such as serial (for example, I2C), parallel, hybrid ports, and the like. Any of the pressure sensors can be part of the wound therapy device or the canister. In some cases, any of the pressure sensors 325 can be remote to the wound therapy device, such as positioned at or near the wound (for example, in the dressing or the conduit connecting the dressing to the wound therapy device). In such implementations, any of the remote pressure sensors can communicate with the I/O module over a wired connection or with one or more transceivers 340 over a wireless connection.

The main controller 310 can receive data from and provide data to one or more expansion modules 360, such as one or more USB ports, SD ports, Compact Disc (CD) drives, DVD drives, FireWire ports, Thunderbolt ports, PCI Express ports, and the like. The main controller 310, along with other controllers or processors, can store data in memory 350 (such as one or more memory modules), which can be internal or external to the main controller 310. Any suitable type of memory can be used, including volatile or non-volatile memory, such as RAM, ROM, magnetic memory, solid-state memory, Magnetoresistive random-access memory (MRAM), and the like.

The main controller 310 can be a general purpose controller, such as a low-power processor or an application specific processor. The main controller 310 can be configured as a “central” processor in the electronic architecture of the control system 300, and the main controller 310 can coordinate the activity of other processors, such as the pump controller 370, one or more communications controllers 330, and one or more additional processors 380. The main controller 310 can run a suitable operating system, such as a Linux, Windows CE, VxWorks, etc.

The pump controller 370 can control the operation of a pump 390, which can generate negative or reduced pressure. The pump 390 can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The pump controller 370 can measure pressure in a fluid flow path, using data received from one or more pressure sensors 325, calculate the rate of fluid flow, and control the pump. The pump controller 370 can control the pump actuator (such as, a motor) so that a desired level of negative pressure is achieved in the wound 104. The desired level of negative pressure can be pressure set or selected by the user. The pump controller 370 can control the pump (for example, pump motor) using pulse-width modulation (PWM) or pulsed control. A control signal for driving the pump can be a 0-100% duty cycle PWM signal. The pump controller 370 can perform flow rate calculations and detect alarms. The pump controller 370 can communicate information to the main controller 310. The pump controller 370 can be a low-power processor.

Any of the one or more communications controllers 330 can provide connectivity (such as, a wired or wireless connection 332). The one or more communications controllers 330 can utilize one or more transceivers 340 for sending and receiving data. The one or more transceivers 340 can include one or more antennas, optical sensors, optical transmitters, vibration motors or transducers, vibration sensors, acoustic sensors, ultrasound sensors, or the like. Any of the one or more transceivers 340 can function as a communications controller. In such case, the one or more communications controllers 330 can be omitted. Any of the one or more transceivers 340 can be connected to one or more antennas that facilitate wireless communication. The one or more communications controllers 330 can provide one or more of the following types of connections: Global Positioning System (GPS), cellular connectivity (for example, 2G, 3G, LTE, 4G, 5G, or the like), NFC, Bluetooth connectivity (or BLE), radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, or the like. Connectivity can be used for various activities, such as pump assembly location tracking, asset tracking, compliance monitoring, remote selection, uploading of logs, alarms, and other operational data, and adjustment of therapy settings, upgrading of software or firmware, pairing, and the like.

Any of the one or more communications controllers 330 can provide dual GPS/cellular functionality. Cellular functionality can, for example, be 3G, 4G, or 5G functionality. The one or more communications controllers 330 can communicate information to the main controller 310. Any of the one or more communications controllers 330 can include internal memory or can utilize memory 350. Any of the one or more communications controllers 330 can be a low-power processor.

The control system 300 can store data, such as GPS data, therapy data, device data, and event data. This data can be stored, for example, in memory 350. This data can include patient data collected by one or more sensors. The control system 300 can track and log therapy and other operational data. Such data can be stored, for example, in the memory 350.

Using the connectivity provided by the one or more communications controller 330, the control system 300 can upload any of the data stored, maintained, or tracked by the control system 300 to a remote computing device, such as the device 334. The control system 300 can also download various operational data, such as therapy selection and parameters, firmware and software patches and upgrades, and the like (for example, via the connection to the device 334). The one or more additional processors 380, such as processor for controlling one or more user interfaces (such as, one or more displays), can be utilized. In some cases, any of the illustrated or described components of the control system 300 can be omitted depending on an embodiment of a wound monitoring or treatment system in which the control system 300 is used.

Any of the negative pressure wound therapy devices described herein can include one or more features disclosed in U.S. Pat. No. 9,737,649 or U.S. Patent Publication No. 2017/0216501, each of which is incorporated by reference in its entirety.

### Multiple Dressing Negative Wound Therapy

FIG. 4 illustrates another negative pressure wound treatment system 400. The system 400 can include a wound therapy device capable of supplying negative pressure to the wound site or sites, such as wound therapy device 110′. The wound therapy device 110′ can be in fluidic communication with one or more wound dressings 406a, 406b (collectively referred to as 406) so as to supply negative pressure to one or more wounds, such as the wounds 104a and 104b. A first fluid flow path can include components providing fluidic connection from the wound therapy device 110′ to the first wound dressing 406a. As a non-limiting example, the first fluid flow path can include the path from the wound dressing 406a to the wound therapy device 110′ or the path from the first wound dressing 406a to an inlet 446 of a branching attachment (or connector) 444 in fluidic connection with the wound therapy device 110′. Similarly, a second fluid flow path can include components providing fluidic connection from the wound therapy device 110′ to the second wound dressing 406b.

The system 400 can be similar to the system 100′ with the exception that multiple wounds 104a and 104b are being treated by the system 400. The system 400 can include any one or more of the components of the system 100′, which are illustrated in FIG. 4 with appended letter “a” or “b” to distinguish between the first and second wounds (such as, the wounds 104a and 104b, the covers 106a and 106b). As illustrated, the system 400 can include a plurality of wound dressings 406a, 406b (and corresponding fluid flow paths) in fluidic communication with the wound therapy device 110′ via a plurality of suction adapters, such as the adapter 108′. The suction adapters can include any one or more of the components of the adapter 108′, which are illustrated in FIG. 4 with appended letter “a” or “b” to distinguish between the first and second wounds (such as, the bridge portions 130a and 130b, the connectors 134a and 134b, and the caps 140a and 140b).

The wound therapy device 110′ can be fluidically coupled via the tube 142 with the inlet 446 of the connector 444. The connector 444 can be fluidically coupled via branches 445a, 445b and tubes or conduits 442a, 442b with the connectors 134a, 134b, which can be fluidically coupled with the tubes or conduits 130a, 130b. The tubes or conduits 130a, 130b can be fluidically coupled with the dressings 406a, 406b. Once all conduits and dressing components are coupled and operably positioned, the wound therapy device 110′ can be activated, thereby supplying negative pressure via the fluid flow paths to the wounds 104a, 104b. Application of negative pressure can be applied until a desired level of healing of the wounds 104a, 104b is achieved. Although two wounds and wound dressing are illustrated in FIG. 4, some implementations of the wound therapy device 110′ can provide treatment to a single wound (for instance, by closing the unused branch 445a or 445b of the connector 444) or to more than two wounds (for instance, by adding branches to the connector 444).

The system 400 can include one or more features disclosed in U.S. Patent Publication No. 2020/0069850 or International Publication No. WO2018/167199, each of which is incorporated by reference in its entirety.

### Canister Status Detection

A negative pressure therapy system can utilize canister status detection system. The canister status detection system can function as a fluid detection system to detect the volume of fluid (or liquid) within the canister (or fill level of the canister) or whether the canister has reached a full or almost full level of fluid (or liquid). One or more alarms or alerts can be generated responsive to the detection. A canister full or nearly full alert can be important for a negative pressure therapy system because it can allow a healthcare professional or a user to replace their canister and continue therapy with the least amount of interruptions (such as, not having to worry about the device suddenly sounding a canister full or canister blockage alarm).

Canister fluid detection systems may rely on comparing peak-to-peak voltage measurements obtained from a pressure sensor to threshold values over a certain period to then trigger the canister full or nearly full alert. In some cases, this approach can be unreliable and can have a low tolerance for variations in conditions. For instance, nuisance alarms may be generated when the canister is empty but there are restrictions in flow from the filter assembly.

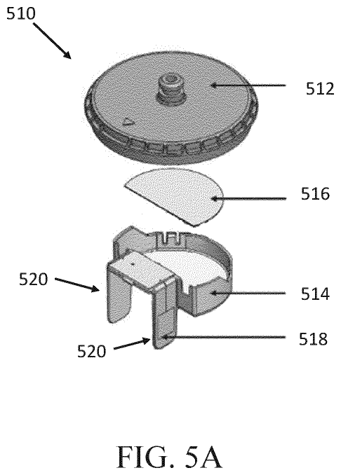

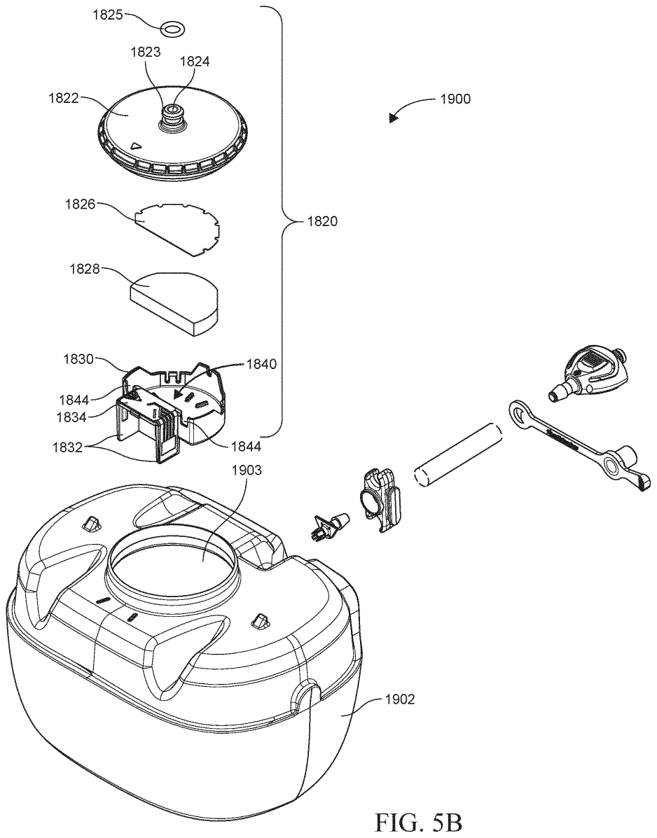

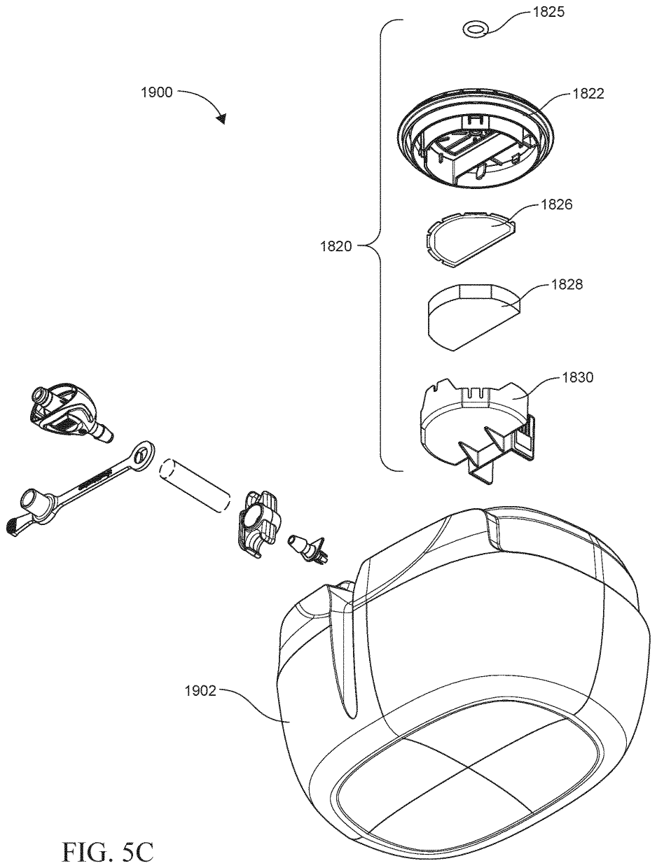

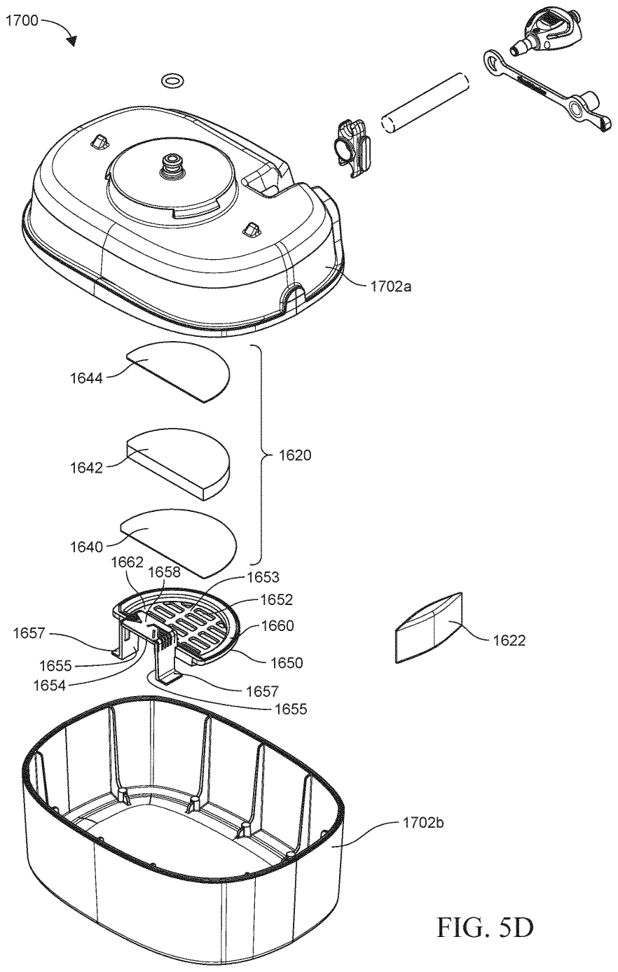

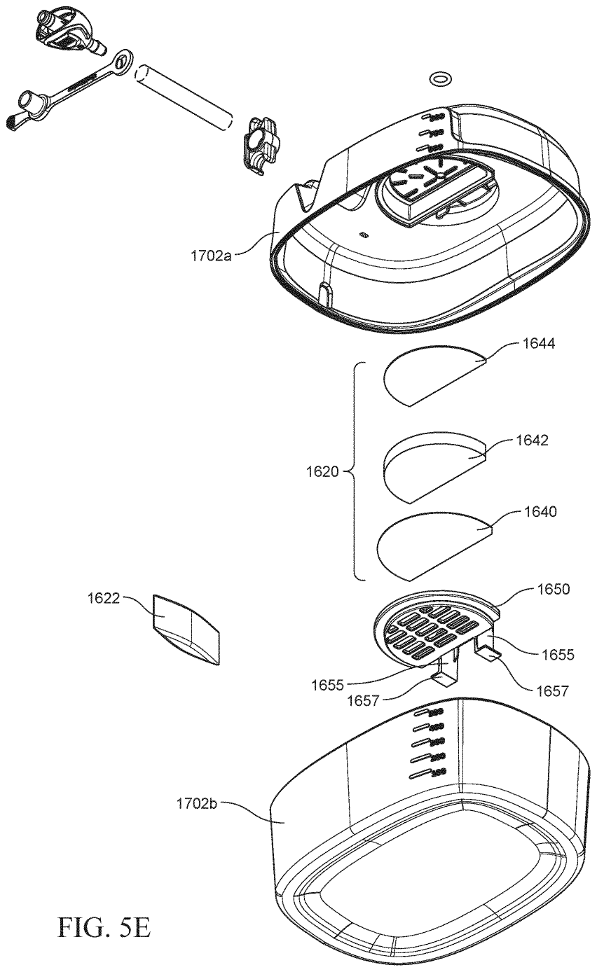

Accordingly, it can be useful to have a more accurate detection method for detecting a full canister condition or a nearly full canister condition that reduces the nuisance alarms. The canister can have a canister detection system that allows for fluid detection that uses a device that communicates with fluid within the canister to detect when the fluid reaches a threshold level within the canister. The canister can incorporate the fluid level sensor device within a surface of the canister. For example, the canister can incorporate a fluid level detector (also referred to as a fluid level sensor) within a cap portion of the canister system. FIG. 5A illustrates a canister cap 510 that can be positioned on a surface of the canister configured to mate or be in a mating arrangement with the negative pressure wound therapy device, such as the pump assembly 160.

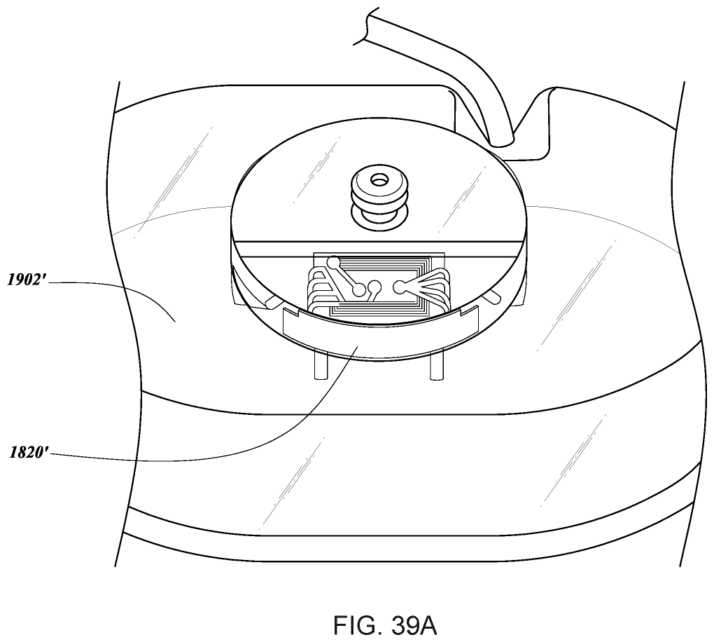

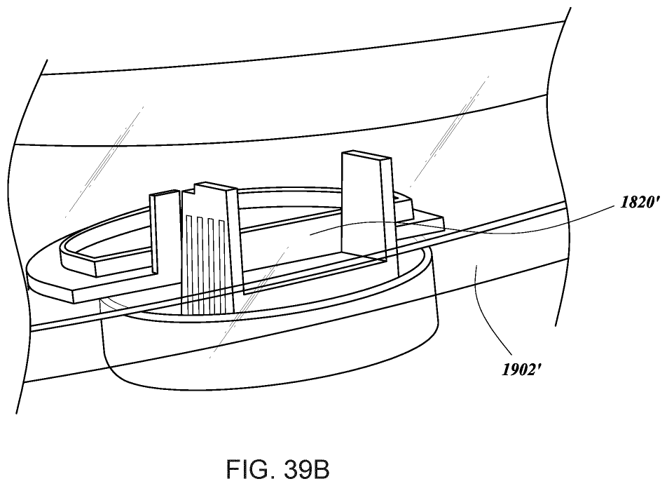

The canister cap 510 can be positioned to provide fluid communication between the negative pressure source and the interior of the canister. For instance, the canister cap 510 can be positioned at the top of the canister 162, as is shown in FIG. 2A. The pump assembly 160 can be removably attached to the canister cap 510. In some cases, the cap can be molded or otherwise be integral with the canister body. That is, the cap can be a component of the canister body. For example, as shown in FIGS. 39A-39B, a cap assembly 1820′ can be moulded as a top portion of a canister body 1902′. The cap assembly 1820′ can be moulded using injection moulding. In some cases, the cap can refer to the cap assembly as defined herein and/or the cap, the canister cap, and/or cap assembly can be any portion of the top of the canister or any surface of the canister and does not have to be a separate component that attaches to the canister housing. The canister cap 510 can include a housing formed from a cap top 512 and a cap bottom 514. The canister cap 510 can include a filter 516 positioned between the cap top 512 and the cap bottom 514. A fluid level sensor 518 can be included within the canister cap 510 to communicate with the interior of the canister. In some cases, the fluid level sensor can include a conductive sensor, an inductive sensor, and/or a capacitive sensors. The fluid level sensor 518 can include two arms 520 that extend from the cap bottom 514 into the interior of the canister. In some cases, the fluid level sensor 518 can be supported by the cap 510 and/or the canister housing. The fluid level sensor 518 can extend from the cap 510 and/or the canister housing into an interior portion of the canister. Although reference is made to the fluid level sensor including two arms 520, the fluid level sensor can include more than or less than two arms (e.g., one arm, three arms, four arms, six arms, etc.). The arms 520 can be made of conductive material (such as, conductive metal). The arms 520 can be used to interact with the fluid within the canister and create a completed circuit and/or cause a state change in the electrical circuit when the arms of the fluid level sensor 518 are in communication with fluid in the canister, which can thereby detect a canister full condition. The fluid level sensor can detect a completed electrical circuit when fluid within the interior of the canister is in contact with the arms of the fluid level sensor. For example, the fluid level sensor can detect fluid collection capacity within the canister when the circuit is open and a canister full condition when the circuit is closed. The arms 520 can be used to interact with the fluid within the canister and create an open circuit when the arms of the fluid level sensor 518 are in communication with fluid in the canister, which can thereby detect a canister full condition. The fluid can cause a normally closed circuit formed by the arms 520 of the fluid level sensor to open. The fluid level sensor can detect an open electrical circuit when fluid within the interior of the canister is in contact with the arms of the fluid level sensor. For example, the fluid level sensor can detect fluid collection capacity within the canister when the circuit is closed and a canister full condition when the circuit is open. A reader within the negative pressure wound therapy device can communicate with the fluid level sensor and, responsive to the canister full condition being detected, can provide an indication of a condition of the canister. The reader can also cause a change in the provision of negative pressure wound therapy (such as, cease application of negative pressure) or cause an alert (such as, a canister full alert) in response to the canister full condition being detected by the fluid level sensor. The reader can be positioned within a housing of the negative pressure wound therapy device, such as the pump assembly 160.

While the fluid level sensor 518 is shown with two downwardly extending arms, the fluid level sensor can include only one arm or any number of arms that extend into the interior of the canister to detect fluid within the canister. In some cases, the fluid level sensor can have any number or extensions or arms as long as at least two separate tracks of conductive material (or electrodes) are present to form the completed electrical circuit.

FIGS. 5B and 5C illustrate an exploded view of a canister cap assembly and the canister body or canister housing with a canister cap assembly 1820 similar to the canister cap and components described with reference to FIG. 5A. The canister cap assembly can be assembled and attached to the canister body which can then be coupled to pump assembly 160 or pump housing as shown in FIG. 2A. The cap assembly 1820 can be configured to be removably coupled (for example and without limitation, threadedly coupled) with an opening (such as 1903 shown in FIG. 5B) of the canister body 1902. In some arrangements, the cap assembly 1820 can be welded to the canister body 1902 or otherwise nonremovably coupled to the canister body. Some arrangements of the cap assembly 1820 can include a cover or first cap member 1822 having a connector interface 1823 that can have an opening 1824 extending axially through a center portion of the first cap member 1822. The connector interface 1823 can project axially away from a first main surface of the first cap member 1822. The connector interface 1823 can have a generally cylindrical shape and an annular flange formed thereon that can be configured to receive a seal, such as an O-ring 1825. The opening 1824 can be configured to provide a fluid passageway for air and/or other gases within the canister body 1902 to pass and to exit the canister body 1902 through.

The cap assembly 1820 can include an upper filter 1826 and an odor filter 1828. The upper filter 1826 can be a hydrophobic filter and/or a dust filter. The odor filter 1828 can also be configured to filter out bacteria from the air flowing through the filter 1828. The upper filter 1826 can be used to prevent any liquids from escaping from the canister body 1902 through the opening 1824 in the first cap member 1822 and can be positioned on either or both sides of the odor filter 1828. The odor filter 1828 can include any suitable filter membrane or material, including carbon. For example and without limitation, some arrangements of the odor filter 1828 can include compressed carbon.

The cap assembly 1820 can also include a base cap support 1830 that can be configured to provide a support surface for one or more of the filters 1826, 1828 and/or other components of the cap assembly 1820. The base cap support 1830 be configured to block or shield the one or more filters 1826, 1828 from exudate and/or other liquids within the canister. In some arrangements, the base cap support 1830 can have a main surface 1840 that can overlap or cover at least a portion of the filter 1828 so as to inhibit or prevent liquid or exudate within the canister 1902 from splashing onto at least a portion of the odor filter 1828 and/or the upper filter 1826. For example and without limitation, the main surface 1840 can overlap at least 80% of a surface area of a lower main surface of the odor filter 1828, or at least 90% of a surface area of the lower main surface of the filter 1828, or from at least 60% or approximately 60% to 90% or approximately 90% of a surface area of the first main surface of the filter 1828.

The base cap support 1830 can have one or more openings 1844 formed therein that air and/or other gases can pass through as the air and/or other gases are being drawn through the cap assembly 1820 when the pump is in operation. The cap assembly 1820 can be configured such that all air or gas or substantially all air or gas coming from the canister body 1802, 1902 must pass through the filter 1828 before passing through the opening 1844 in the cap assembly 1820. In some arrangements, there can be 3 or more, 4 or more, 5 or more openings 1844 formed in the base cap support 1830. The openings 1844 can be formed in walls that are perpendicular to a top main surface of the canister body 1802, 1902 so that exudate is less likely to splash or otherwise pass through the openings 1844—e.g., the openings 1844 can be formed in vertical walls of the base cap support 1830.

The cap assembly 1820 can include a fluid level sensor 1834 to detect the fluid level within the canister and/or if the canister is full or nearly full. The fluid level sensor 1834 can include two downwardly extending arms 1832. The fluid level sensor 1834 and arms 1832 can be similar to the fluid level sensor 518 and arms 520 described with reference to FIG. 5A.

FIGS. 5D and 5E illustrate an exploded view of a canister assembly 1700. The canister assembly 1700 can have a canister body 1702 with a first body portion 1702a and a second body portion 1702b. The canister assembly can include a filter assembly 1620. The filter assembly 1702 can include a hydrophobic filter 1640, an odor filter 1642, and a dust filter 1644 that can be used to inhibit (e.g., prevent) dust or other particulates from passing through to the pump assembly. The odor filter 1642 can also be configured to filter out bacteria from the air flowing through the filter assembly 1620. The hydrophobic filter 1640 can be used to prevent any liquids from escaping from the canister body 1702 and from contacting the odor filter 1642. The odor filter 1642 can include any suitable filter membrane or material, including carbon. For example and without limitation, some arrangements of the odor filter 1642 can include compressed carbon. The filter assembly 1620 is also shown in FIGS. 5D and 5E.

The filter assembly 1620 can be supported at a lower end or interior end by a base support 1650 that can be configured to provide a support surface for the hydrophobic filter 1640 and/or other components of the filter assembly 1620. The base support 1650 can have one or a plurality of openings 1652 through a main surface 1653 thereof through which air/or other gases can pass as air and/or other gases are being drawn by the pump through the filter assembly 1620.

Some arrangements of the base support 1650 can optionally be configured to support a sensor or sensors and/or other electronics components. With reference to FIG. 5D, some arrangements of the base support 1650 can have a support surface 1654 configured to support a sensor 1658 and/or other electronic components. For example and without limitation, the base support 1650 can have a support surface 1654 that is approximately parallel with a top surface of the canister assembly 1700. In some arrangements, the base support 1650 can also have one or more support tabs 1655 (two being shown) to provide additional support for a sensor or sensors and/or other electronics components. For example, the sensor can include a pair of electrodes configured to determine a fill level of the canister or detect that the canister is full responsive to a detection of electric current being conducted between the electrodes via liquid (e.g., wound exudate) aspirated into the canister as described herein. The support tabs 1655 can support the pair of electrodes, which can be positioned on the outward facing side of the support tabs 1655.

The support tabs 1655 can extend away from the support surface 1654 toward a bottom of the canister. The support tabs 1655 can have a flange or shield 1657 at a distal end of each of the support tabs 1655 to inhibit liquid (e.g., wound exudate) within the canister from splashing onto the support tabs 1655 and/or the electronics components 1658 (such as, electrodes) and from exposure to a gel packet 1622 or a mound of gelling agent. In some arrangements, the flanges 1657 can each extend at an angle (e.g., at a perpendicular angle) away from the support tabs 1655. In other arrangements, the flanges 1657 can extend at an angle that is greater than or less than 90 degrees relative to the support tabs 1655.

In some arrangements, the electronics components 1658 can optionally be a fill level sensor or a canister full sensor as described herein. For example, the fill level sensor can have a wireless transmitter thereon (that can optionally be a near field communication transmitter) that can be configured to communicate status information (such as, detected fill level or whether the canister is full) to a wireless receiver in the pump assembly or otherwise, or can have a wired connection through the canister in communication with the pump assembly. The flanges or shields 1657 can reduce or prevent fluid from splashing onto the fill level sensor or canister full sensor to prevent false detection. In some cases, the flanges or shields 1657 can be utilized to shield the electronics from the gelling agent bag or fluid solidifier within the canister. The fill level sensor or canister full sensor can be adhered to or otherwise fixed or attached to the support surface 1654 and/or the support tabs 1655. In other cases, the fill level sensor or canister full sensor can rest on at least a portion of the support surface 1654 and/or the support tabs 1655.

The base support 1650 can have an annular flange 1660 around a perimeter thereof and a recessed portion 1662 that can be configured to receive and support at least the hydrophobic filter 1640. The base support 1650 can be welded, adhered, or otherwise coupled within an inside surface of the first body portion 1702a of the canister body 1702 of the canister assembly 1700, optionally, before the first and second portions 1702a, 1702b of the body 1607 are coupled together.

The fill level sensor or canister full sensor can be overmoulded into the canister assembly. In other cases, the fill level sensor or canister full sensor can be insert moulded into the canister assembly. The fill level sensor or canister full sensor can be screen printed onto the canister assembly, for example, the electrical tracks can be screen printed onto the canister assembly. These techniques can be helpful during assembly and manufacture because these methods can eliminate the need to put the fill level sensor or canister full sensor into the canister. The fill level sensor or canister full sensor can be adhered to the support surface and/or the support tabs by ultrasonic welding or an adhesive. This method can take advantage of manufacturing methods for ultrasonic welding and adhesive application that are used for other components of the canister.

The fluid level sensor can include or be part of a detection system configured to communicate wired or wirelessly (such as, using NFC, RFID, etc.). The detection system can utilize fluid level sensor that incorporates a communication device to communicate information from the canister to the device or another remote computing device 334 (such as, the remote computing device 334). The detection system can utilize a communication device for communicating the information from the canister to the device using NFC. NFC is a set of short-range wireless technologies, typically requiring a separation of 10 cm or less (in some cases, 4 cm or less). NFC can involve an initiator (or active tag) and a target (or passive tag). The initiator can actively generate a radio frequency (RF) field that can power the passive tag. In some cases, NFC communication can utilize an NFC reader communicating with a passive NFC tag. The NFC reader can retrieve information stored in the passive NFC tag. The pump assembly 160 can include an NFC reader (which can be located at or near the bottom of the pump assembly 160 housing) and the detection system of the canister can include a passive NFC tag. When the conductive portions of the NFC tag are in contact with fluid within the canister and the circuit is closed, information (such as, a flag) can be stored in memory of the NFC tag. The NFC reader can read such information by communicating with the NFC tag. The NFC reader and NFC tag can include one or more antennas to facilitate wireless communication (for example, facilitate transmission and reception of data). In some cases, the range of communication between the NFC reader and the NFC tag can be about 20 mm (or less or more), which can exceed the distance between the NFC reader and the NFC tag. Additional details of utilizing NFC communication for determining canister status are disclosed in: co-pending International Patent Application No. PCT/EP2022/060464 (Atty. Docket SMNPH.654WO) titled “COMMUNICATION SYSTEMS AND METHODS FOR NEGATIVE PRESSURE WOUND THERAPY DEVICES,” filed on Apr. 20, 2022; co-pending International Patent Application No. PCT/EP2022/060463 (Atty. Docket SMNPH.672WO) titled “CANISTER STATUS DETERMINATION FOR NEGATIVE PRESSURE WOUND THERAPY DEVICES,” filed on Apr. 20, 2022; co-pending U.K. Application No. 2205753.3 (Atty. Docket SMNPH.712 GB) titled CANISTER STATUS DETERMINATION FOR NEGATIVE PRESSURE WOUND THERAPY DEVICES, filed on Apr. 20, 2022; co-pending U.K. Application No. 2214052.9 (Atty. Docket SMNPH.715 GB) titled CANISTER STATUS DETERMINATION FOR NEGATIVE PRESSURE WOUND THERAPY DEVICES, filed on Sep. 27, 2022; and co-pending U.K. Application No. 2214074.3 (Atty. Docket SMNPH.717 GB) titled PROTECTION AND ISOLATION OF SENSING CIRCUITRY FOR NEGATIVE PRESSURE WOUND THERAPY DEVICES, filed on Sep. 27, 2022, the disclosures of which are hereby incorporated by reference in their entireties.

As described herein, a negative pressure wound therapy device, such as the pump assembly 160, or a remote computing device can include an NFC reader. The NFC reader can have an antenna configured to facilitate communication with the canister, such as the canister 162. The pump assembly 160 can receive data relating to the status of the canister, such as whether the canister is full, the level of fluid in the canister, or the like. Additional approaches for communicating the with the pump assembly are disclosed in the above-referenced International Patent Application No. PCT/EP2022/060464 (Atty. Docket SMNPH.654WO) titled “COMMUNICATION SYSTEMS AND METHODS FOR NEGATIVE PRESSURE WOUND THERAPY DEVICES.”

To detect the fluid level within the canister, the detection system can utilize a tamper detection system to detect when the fluid in the canister reaches a threshold level. The tamper detection system can detect a change in impedance or resistance in the circuit to determine if the system has been tampered with. For example, when used in packaging, detection of an open circuit in the tamper detection system can indicate tampering of the device such as the opening of a parcel. The fluid detection system can utilize the tamper detection system to detect canister full when the tamper detection system detects a closed circuit caused by the fluid level in the canister reaching (or exceeding) a threshold fluid level. Accordingly, the tamper detection system can detect canister full responsive to a closed circuit, which can be indicative of no tampering (rather than tampering detected with the open circuit as used in the case of packaging solutions).

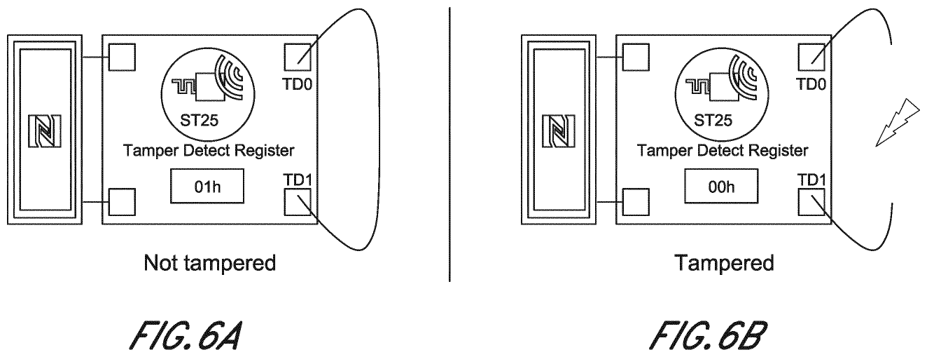

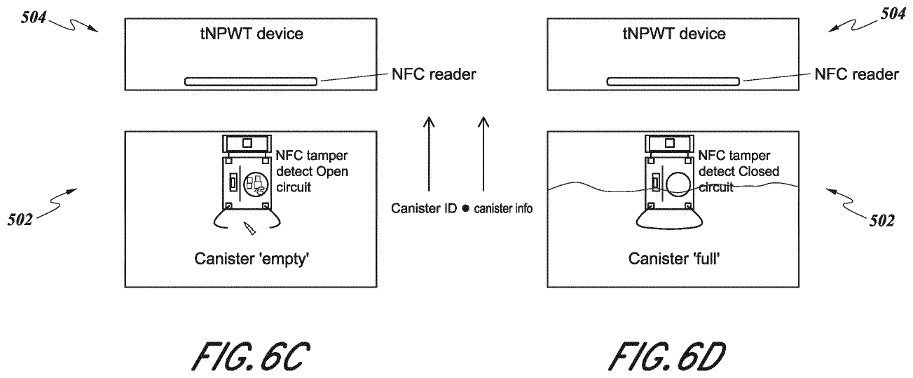

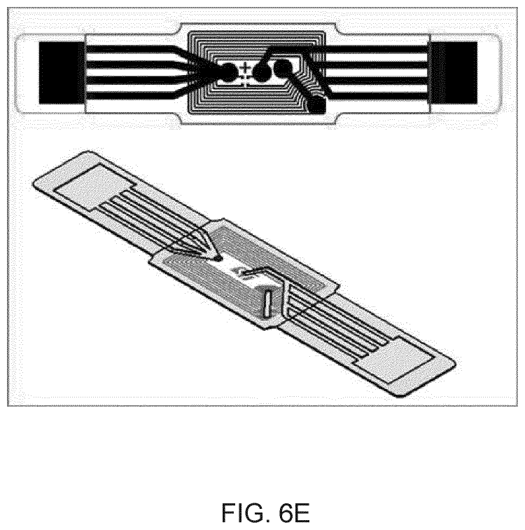

FIGS. 6A and 6B illustrates an example tamper detection system circuit and the state of tamper detection circuit. The illustrated tamper detection system can be configured to communicate using NFC (using an antenna illustrated on the left). FIG. 6A illustrates the tamper detection system in a “not tampered” state corresponding to a closed circuit state (in which nodes TD0 and TD1 are electrically connected). FIG. 6B illustrates the tamper detection system in a tampered state or open circuit state (in which nodes TD0 and TD1 are not electrically connected). The state of the system can be stored in a dedicated register. For instance, “01 h” (or “1”) can indicate closed circuit and “00 h” (or “0”) can indicate open circuit. The fluid within the canister can be used to close the circuit creating the not tampered state illustrated in FIG. 6A. When the tamper detection system is incorporated into a fluid detection system (such as, the fluid level sensor 518), the fluid detection system can have a first state that can be the ‘canister empty’ condition that corresponds to an open circuit (such as, ‘tampered’ as shown in FIG. 6B), and a second state can be a ‘canister full’ condition that corresponds to a closed circuit due to the fluid within the canister completing the circuit (such as, ‘not tampered’ as shown in FIG. 6A). For example, when fluid within the canister reaches a certain level, the arms 520 of the fluid level sensor 518 can be in contact with the fluid within the canister thereby completing the circuit and allowing the fluid level sensor to detect a canister full condition.

The tamper detection system can detect that the circuit is closed based on monitoring the impedance or resistance between nodes TD0 and TD1. In some cases, a threshold impedance for detecting a closed circuit should correspond to the impedance of the fluid (such as, wound exudate) expected to fill the canister. Testing of various fluids, including tap water, saturated salt water, and stimulated exudate (0.9% saline), has revealed impedances of about 460Ω, about 34Ω, and about 66Ω, respectively. The threshold impedance can be set to a value that equals or exceeds these impedances. For instance, the threshold impedance can be set to about 35Ω (or more), about 50Ω (or less or more), about 70Ω (or less or more), about 10052 (or less or more), about 0.5 kΩ (or less or more), about 1 kΩ (or less or more), about 2 kΩ (or less or more), about 3 kΩ (or less or more), about 4 kΩ (or less or more), about 5 kΩ (or less or more), about 6 kΩ (or less or more), or less than about 7.5 kΩ. In some cases, the threshold impedance can correspond to an average impedance of at least some of the fluids expected to fill the canister.

In some instances, the threshold impedance can be a preset value. The area of one or more of the electrodes of the fluid level sensor can be adjusted to ensure that the impedance of the closed circuit matches such present threshold impedance. For example, suppose that the preset threshold impedance is no more than 50Ω. In such case, the area of the one or more of the electrodes can be increased to ensure that closed circuit impedance does not exceed 50Ω.

In some cases, a reliable canister detection can be implemented. The fluid detection system can communicate an identifier, which can be a unique value (such as, a unique canister identifier). For instance, when NFC protocol is used (which operates over a short range), the negative pressure wound therapy device can be configured to disallow provision of therapy unless the identifier has been received from the canister (indicating that the canister has been attached to the device).

In some cases, the pump assembly can be used with canisters of various volumes or sizes. In such cases, the canisters of different volumes or sizes can be identified by changing the location (or number) of the fluid level sensor (for example, NFC tag) within the canister on the different types of fluid containment canister. For example, a canister of a first size can have a fluid level sensor or NFC tag (which may be in a first location, such as the center top of the canister body) while a second canister of a second size can have a fluid level sensor or NFC tag (which may be positioned in a second location, such as the top side of the canister body). The location of the reader may be remote of the first or second location. The reader of the pump assembly can identify or can determine the type of canister that has been attached to the pump assembly, rather than reading a coded message on the sensor to determine the canister type.

Additionally, using sensors positioned at different locations for canisters for determining the canister volumes or sizes can allow for identification of the canister type even if data written to the sensor is written incorrectly. In some cases, larger canisters can include more sensors than smaller canisters. For example, a small canister can include one sensor positioned at a first location and configured to identify the presence of a fluid. A medium canister, larger than the small canister, can include two sensors. The first sensor can be positioned at the same or similar location as the sensor of the small canister and the second sensor can be positioned on a second location. The first and second sensors can be configured to identify the presence of a fluid at different location of the canister. For example, each location can identify a fill volume (e.g., 300 ml, 500 ml). A large canister, larger than the small and medium canister, can include three sensors. The first sensor can be positioned at the same or similar location as the sensor of the small and medium canisters, the second sensor can be positioned at the same or similar location as the second sensor of the medium canisters, and a third sensor can be positioned at a third location. The first, second, and third sensors of the large canister can be configured to identify the presence of a fluid at different locations of the canister. For example, each location can identify a fill volume (e.g., 300 ml, 500 ml, 800 ml). The use of more than one sensor can beneficially provide an indication that one or more of the sensors are failing/not working properly. For example, a sensor reading a fill volume larger than the fill volume of a second sensor and/or a second sensor failing to read the smaller fill volume would provide an indication that at least one of the first and second sensors are not working properly.

In some cases, the canisters of different volumes or sizes can include one or more physical markers. The canisters of different volumes or sizes can be identified by changing the location (or number) of the physical markers within the canister on the different types of fluid containment canister. For example, a canister of a first size can have a first physical marker in a first location (such as the center top of the canister body) while a second canister of a second size can have a physical marker positioned in a second location (such as the top side of the canister body). The first and second physical markers can be identified by the reader on the pump assembly to indicate the type of canister that has been attached to the pump assembly, rather than reading a coded message on a sensor to determine the canister type.

In some cases, the canisters of different volumes or sizes can include one or more markers. The marker can include a conductive marker such as a metal marker. The one or more markers can include different shapes, sizes, and/or materials, and be placed on different locations of a canister. The canisters of different volumes or sizes can be identified by changing the location, number, shape, size, and/or material of the marker within the canister on the different types of fluid containment canister. For example, a canister of a first size can have a first marker including a first shape and size in a first location (such as the center top of the canister body) while a second canister of a second size can have a marker including a second size and shape in a second location (such as the top side of the canister body). The first and second markers can be identified by electrical sensing shielding and/or by an NFC antenna.

The state of the tamper detection system 502 that supports NFC communications (such as, functions as a passive NFC tag) can be detected with an NFC reader 504 as illustrated in FIG. 6C-6D. As described herein, the NFC reader 504 can be located within the negative pressure wound therapy device (labeled as “tNPWT device” in FIGS. 6A-6D), such as the pump assembly 160. Additional details about how the NFC reader can be integrated within the negative pressure wound therapy device are disclosed in the above-referenced International Patent Application PCT/EP2022/060464 (Atty. Docket SMNPH.654WO), titled “COMMUNICATION SYSTEMS AND METHODS FOR NEGATIVE PRESSURE WOUND THERAPY DEVICES.”

In some cases, the tamper detection system can include memory that stores additional information related to the status of the canister. For example, the additional information may include information about the canister size (such as, 300 ml or 800 ml). In some cases, the additional information can include an indication whether the same canister had been previously attached to the negative pressure wound therapy device. For example, a unique canister identifier can indicate if the same canister is being removed and put back on the negative pressure system. The unique canister identifier can allow for tracking of the use of the canister. In some cases, the additional information stored in the tamper detection device in the canister can include tracking information entered into the device, such as the time and/or date when the canister was connected to the pump assembly, when the canister was removed from the pump assembly, and/or the number of hours of operating time. Additional details of the status of the canister are disclosed in International Patent Application No. PCT/EP2022/060459 (Atty. Docket SMNPH.683WO), titled “INTELLIGENT DISPOSABLE DEVICES FOR WOUND THERAPY AND TREATMENT,” filed on Apr. 20, 2022, and incorporated by reference in its entirety.

In some cases, the tamper detection system can be used in combination with other canister full detection methods. Using two or more redundant systems can promote accuracy. For example, the tamper detection system can be used in combination with using a peak-to-peak voltage measurement from pressure pulses to avoid false positive alarms from the peak-to-peak voltage measurement system. This can be particularly advantageous when estimating the fill rate of the canister. For example, in some cases, the peak-to-peak voltage measurements can give a crude indication of the fill level and therefore the flow rate of the wound drainage. The tamper detection system could be used to ensure the alarm will trigger when the canister is near a full level or is full. Further, a third check by the user could be implemented to increase accuracy of the system. This third check can consist of a user interface input of the canister level (for example, ‘low’, ‘half-full’, ‘nearly full’). In some cases, the user interface can utilize buttons or other modes of user input, as described herein. For example, pictures or text can be used on the buttons (e.g. using three buttons with each corresponding to a certain fluid level-each with a picture and/or text).

The fluid detection system can include a fill level sensor or canister full sensor that includes multiple sets of electrode pairs. In some cases, the multiple sets of electrode pairs can be positioned on multiple arms. Additionally or alternatively, the fluid detection system can utilize multiple fill level sensors or canister full sensors (for example, multiple NFC tags) within the canister. For example, in some cases, rather than a single pair of arms or a single sensor extending into the canister as shown in FIG. 5A, multiple pairs of arms or multiple sensors can be used. Multiple pairs of arms or multiple sensors could be used with different lengths to measure a fluid level as the canister fills with liquid and before it becomes full or nearly full. In some cases, the sensor as described herein can include multiple pairs of arms at multiple distance from the canister top or cap. In some cases, multiple sensors can be used and stacked or positioned in an arrangement that allows for them to detect at different fill levels as the canister fills with liquid. This can allow for detection at various levels within the canister to allow the user to determine the amount of volume remaining the canister or to determine a fill rate of the canister as described herein.

For example, there can be three pairs of arms positioned at or extending into the canister body to different locations within the canister. A first pair of arms (longest pair of arms) can extend into the canister to a position the would indicate the canister is half filled with liquid. A second pair of arms can be shorter than the first pair but longer than the third pair and can extend into the canister to a position between a position that would indicate the canister is half filled and a position that would indicate the canister is full. A third pair of arms can be the shortest pair of arms and is positioned in a location that would indicate the canister is full or nearly full. If only the first pair of arms detect fluid, then the canister is half full. If the second pair of arms detect fluid then the canister can be ¾^{th} of the way full or some volume between halfway full and full or nearly full. If the third pair of arms detect fluid, then the canister is full or nearly full. The multiple pairs of arms can also be used to measure a “time to full” of the canister as described further herein. The pairs of arms can be on the same sensor device (or same support substrate) or can be in the form of multiple sensors (for example, multiple NFC tags) each having pairs of arms and each pair of arms differs in length from another pair of arms. While three pairs of arms are described, any number of pairs of arms or any number of sensor devices can be used to detect any number of levels within the canister. The fill level resolution can be increased with more sensors or pairs of arms at various levels. In some cases, a single support substrate can include two or more conductive traces. In some cases, a sensor can include two substrate arms. Each of the substrate arms can include a conductive trace. Sensors including two substrate arms with each arms having a conductive trace can be replaced with a sensor having a single substrate arm with two or more conductive traces.

In some cases, multiple sensor devices can be positioned at the same distance from the filter in the canister top but in multiple axes. The multiple sensors can generate redundancy against failure of a tag. Multiple sensors also offer the capability to mount the sensors offset to generate a fill gauge. The spacing of the tag read paths to generate the fill gauge can be set to a fixed increment of fluid or in a spacing appropriate for an expected time increment (e.g. wider spaced towards the start or as the bottom of the canister fills to cope with a bolus of fluid delivered to the canister and then the tags can be more closely spaced later as it would be expected that the fluid rate tails off as the canister fills). The latter would give the greatest granularity of whether the wound is following an ‘expected’ path with the minimum number of track paths.

FIG. 6E illustrates an NFC tag that can be used as a fill level sensor assembly. The NFC tag can be used for detecting the fluid level within the canister as described herein. The canister can be a critical portion of the negative pressure therapy. In some cases, pump assembly cannot perform patient negative pressure therapy without the canister present. The canister must be pneumatically connected and attached to the pump assembly and/or a wound dressing. Any of the canister cap embodiments described herein can contain the NFC tag fill level sensor assembly as shown in FIG. 6E. In some cases, as described herein, the short-range wireless NFC communication can validate the attachment of the canister to the pump assembly and validate that the canister is attached properly. The main function of the canister's NFC tag fill level sensor assembly is to allow the passive NFC device within the canister to wirelessly communicate to the NFC reader located in the pump assembly. The wireless function of the NFC tag sensor assembly can transmit data relating to canister detection, fluid detection, and programmed canister data. The NFC tag device illustrated in FIG. 6E includes the tamper-proof feature described here. In some cases, the NFC tag device can include specific modes to protect tag access, such as an untraceable mode. In some cases, the NFC device can include a digital signature used to prove the origin of the chip in cloning detection, embed a configurable EEPROM with 60-year data retention, and/or can be operated from a 13.56 MHz long-range RFID reader.

### Canister Fill Rate Detection

It can be advantageous to detect and indicate the fill rate of a canister, such as the canister 162, in the negative pressure system. This detection can be performed in addition to or as an alternative to the canister full or near full detection described herein.

The canister fill rate can be used to infer the wound drainage or how much exudate is flowing out of the wound. The flow rate of fluid from the wound can be calculated using the time taken to fill the canister based on the duration of time between an empty canister being installed and the time canister full is detected. The volume of the canister can then be divided by the time taken to fill the canister to calculate the flow rate of wound fluid. For example, the following equation can be used:

flow rate=(canister volume)/(time taken to fill) (1)

In some cases, the volume of the canister can be input by the user or can be detected (such as, retrieved from the memory of the canister as described herein). An accurate flow rate can be calculated using the fluid level sensor described herein. Time duration for filling the canister can be accurately determined by taking into account any stoppages or pausing of therapy, determination that the same canister is still attached there when therapy is resumed, and determination that the canister has been removed and reapplied. As described herein, these determinations can be made using the unique canister identifier.

In some cases, the flow rate can then be used by the healthcare provider to determine the best route of care and if the patient can be moved to another system, for example a Pico Single Use Negative Pressure Wound Therapy System from Smith & Nephew.

In some cases, the canister can be removed before the canister is full. In these situations, an inference can be made about the flow rate by providing a manual input to the user interface to state the level of fluid within the canister. In some cases, this input could be an input noting: ‘low’, ‘half-full’, ‘nearly full’, if the canister is removed before the canister is full or before the canister full alarm is triggered. This input can be used to adjust the canister volume variable in Equation 1.

In some cases, the information obtained from the canister, the canister fill rate, and/or canister use and therapy use can provide information to the healthcare practitioner and to allow for an understanding of trends and behaviors for a single patient or among various patients to assist in making healthcare decisions.

In some cases, a flow meter can be positioned in the fluid flow path (such as, in the conduit 108 or 108′) to determine the flow rate. For example, a venturi tube can be used.

### Fluid Level Sensor Configurations

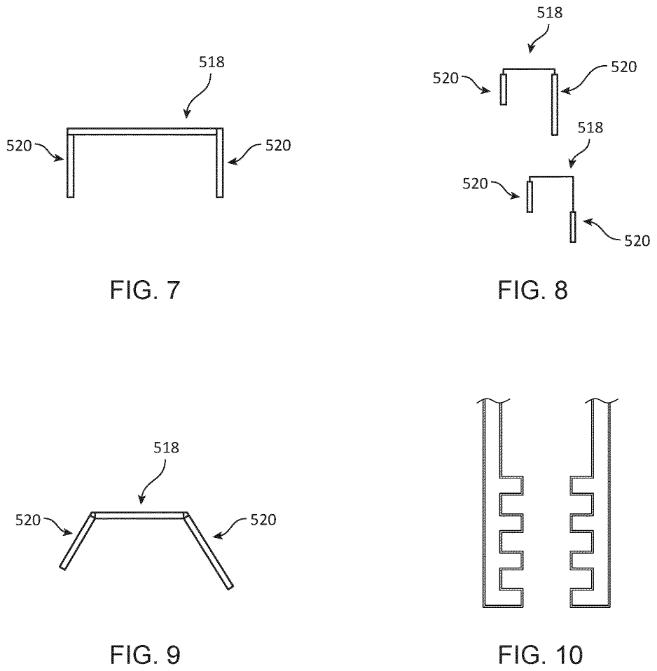

The arms 520 of the fluid level sensor 518 can be configured in different ways. For example, in some cases, the fluid level sensor 518 can include two arms 520. Each arm can comprise a planar pathway surface. In some cases, the planar pathway surfaces of the arms 520 face each other, as shown in FIG. 7. The arms 520 of the fluid level sensor 518 can also comprise planar pathway surfaces parallel to each other. The planar pathway surfaces of the arms 520 can also be coplanar. That is, each planar pathway surface of the arms 520 can lie substantially on the same plane. In other cases, the fluid level sensor 518 can include two offset arms 520, that is, two arms 520 of different lengths, as shown in FIG. 8. The length of the arms can comprise a distance between two points A and B. Point A can be a point located along the canister top and Point B can be a point furthest away from the canister top. The arms 520 can comprise planar pathway surfaces parallel to each other and/or coplanar to each other. In yet other cases, the fluid level sensor 518 can include two arms 520 of similar or different lengths wherein the arms 520 are angular to each other, as shown in FIG. 9. In some cases, the arms 520 of the fluid level sensor 518 are not mechanically fixed in place. In such cases, the position of the arms 520 inside the canister 162 can vary according to the orientation of the canister 162. The distance between the arms 520 can also vary. In some cases, the arms 520 can be closer to each other than in other cases. Beneficially, the different configurations of the arms 520 can improve the ability of the fluid sensor 518 to accurately detect a state of the fluid level sensor 518 even when the canister 162 has been tipped or is not in the upright position. The canister 162 of the negative pressure wound therapy device can be shaped so that the canister 162 can sit in one or more positions. The fluid sensor 518 can be positioned to detect a condition (e.g., canister full condition) notwithstanding the position in which the canister 162 sits.

One of the important features of the fluid sensor can be to alert the user when the canister is full or nearly full and therefore prevent the fluid from blocking the filter which would lead to loss of negative pressure. The movement of a portion of the sensor that is not mechanically fixed in place under gravity would allow the sensor to provide a ‘full’ indication at the same (or similar) point below the filter in multiple orientations. Additionally, if the canister were inverted, the fluid sensor that is not mechanically fixed in place could provide a warning to the user. This warning could occur if the arms of the fluid sensor were to touch the top of the canister where they were contacted by any fluid and/or a conductive path on the canister itself.

In some cases, the negative pressure wound therapy system includes a single fluid level sensor 518 having one or more sets of arms 520, each set of arms 520 configured to detect a condition of the fluid level sensor 518. In some cases, the negative pressure wound therapy system includes one or more fluid level sensors 518. Each fluid level sensor 518 of the one or more fluid level sensors 518 can include one or more sets of arms 520 configured to detect a condition of the fluid level sensor 518. The multiple detection points within the canister achieved with the multiple sensors or multiple sets of arms 520 can also be achieved by utilizing a fluid level sensor 518 with one common long arm. For example, detecting fluid at multiple levels can be achieved with one common long arm, and multiple arms of differing length to which it looks for a connection.

In cases where the fluid level sensor 518 includes an NFC tag, a single NFC tag having a single antenna and a single chip (e.g., IC chip) can include multiple pathways disposed at different elevations and/or locations inside the canister 162 for detecting different conditions inside the canister 162. In some cases, a single NFC tag can include a single pathway for detecting a condition inside the canister 162. The negative pressure wound therapy device can include multiple NFC tags. In cases where multiple NFC tags are used, multiple NFC readers, a moving (e.g., rotating, oscillating) single NFC reader or a multifrequency NFC reader can be used with channels at different frequencies (e.g., 3.56 (+/−0.9) MHz plus an 865-868 or 902-928 MHz—e.g., following EPC Gen2 protocol).

### Fluid Characteristic Detection

In some cases, different techniques and types of sensors for granular and continuous fill rate measurement can be used. These techniques and types of sensors can allow for an alert when there is a change of flow rate or change of composition of fluid. The granular and continuous fill rate measurement can be monitored using an analogue device. For example, an analogue pathway (rather than a digital measurement) can be used to determine the status of the fluid in the canister and fill rate. The analogue pathway can be used on a conductive, inductive, and/or capacitive path. As the canister fills the fluid within the canister can contact the arms and the conductive, inductive, and/or capacitive path on the arms of the sensor device as described herein. For instance, as the fluid contacts the arms, the conductivity reading of the paths would increase since the wound exudate fluid is more conductive than air within the canister. Accordingly, the conductivity measurement would continue to increase as the canister fills with wound fluid because more fluid within the canister will contact more surface area of the arms. In some cases, as the fluid contacts the arms, the fluid could create a conductive path across a shorter path. The shorter path can reduce the distance through the arms that the current needs to pass therefore reducing the circuit impedance. As another example, the impedance in the path can be measured as the canister fills with fluid. The impedance can decrease as more fluid contacts the surface area of the arms and the fluid contacts more of the path.

As yet another example, the capacitance of the paths can be measured as the canister fills with fluid. Suppose that more fluid contacts the surface area of the arms and the fluid contacts more of the path. In this example, at least one of the tracks or paths can have a non-conductive coating (or can be on the outside of a non-conductive separator). For instance, there can be only a single track into the fluid with the other side of the circuit isolated. As the fluid covers the arms, the capacitance will increase because there is a greater area of charge coupling into the conductive fluid. High capacitance may necessitate having a high direct current (DC) impedance (or resistance).

The analogue sensor can communicate with a reader as described herein. The sensor could generate a multi-bit value from the path as opposed to a single binary bit on the path that can be generated with a sensor such as the NFC sensor system described herein. Such multi-bit value can be indicative of the level of fluid in the canister. The reader which receives data from the sensor could therefore receive the multi-bit value generated from the path. For example, in such cases, the sensor can provide a reading of capacitance or conductance and based on that reading the fluid level can be determined. This allows for more granularity and a continuous fill rate measurement rather than just a reading of if the canister is full or not full.

As described herein the arms or electrodes of the sensor device with analogue read paths can be any shape or configuration described herein. In some cases, the electrodes can be in the configurations described herein but can include a non-continuous shape or a shape that changes along the length of the electrode (measured from a first end portion within or connected to the canister cap to a second end of the electrode as it extends into the canister) as illustrated in FIG. 10. The electrode with non-continuous shape or changing shape along the length of the electrode can have a parallel, perpendicular, and coplanar configuration or any other configuration described herein with the electrodes or sensor arms described herein. In some cases, the electrodes can be a wedge shape and/or other shaped electrodes. The wedges and/or other shapes can allow for a change of sensitivity. For example, the larger the electrode at a given depth of fluid, the higher the sensitivity to changes of fluid height.

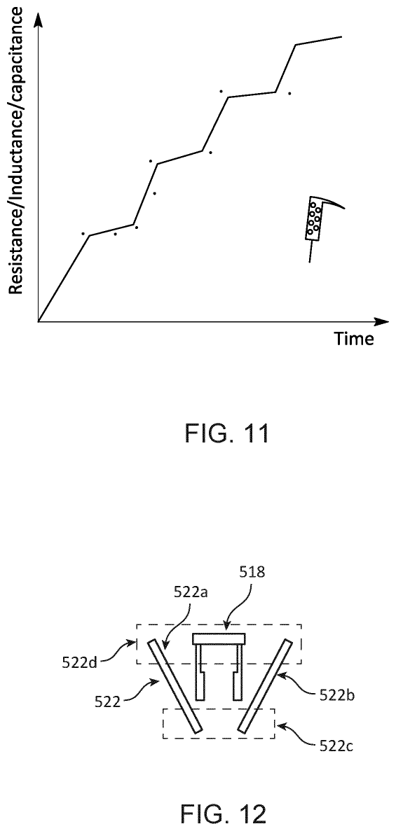

A continuous fill of homogenous fluid can generate a rate conductive/inductive/capacitive graph as shown in FIG. 11. Time-base averaging and/or elimination of top/bottom values over a time base can be used to remove slosh effects of the canister. Because of the shape of the electrode as shown in FIG. 10, each inflexion point of the graph shown in FIG. 11 would identify an increment on the fill volume. Between each inflexion the lines would be straight if the fluid flow rate was continuous and the fluid within the canister did not change. A curvature of the line (i.e. a change in differential of the line) can indicate either a change of flow rate or of the composition of the fluid. At the next inflexion point it would be possible to identify whether it was a flow rate issue or composition of the fluid. Alerts/alarms can occur either at any change of differential (curvature of the line) and/or when the inflexion point is achieved, and which cause was identified. The ratio of the differentials of the line between different inflexion points can indicate a validation of the sensor function (for example, because the size of the contact is known the ratio of gradients should change by a fixed amount if the sensor is operating correctly). In some cases, the system can include the inclusion of an IMU (inertial measurement unit). The IMU value can provide the system with information relating to the movement of the canister. The system can use the IMU value to only read or include readings when the canister is not moving.

In some cases, the viscosity or other characteristics of the fluid in the canister can be determined based on monitoring the interaction of the fluid with a sensor in the canister. For example, a single sensor can be used to monitor how the sensor is covered or uncovered with fluid over a period of time. The covered and uncovered determinations can create a unique identifier of characteristics of the fluid. For example, fluid running over a weir shape with beyond-vertical drop or flowing over a splitter can be used to determine viscosity of the fluid.

In some cases, the viscosity or other characteristics of the fluid in the canister can be determined based on monitoring the interaction of the fluid with at least two sensors in the canister over a period of time. There can be at least one sensor path at a first position in the fluid flow path in the canister (for example, at an input of the canister) and at least one sensor path at a second position in the fluid flow path in the canister (for example, at a lower point on the canister). The contact of the fluid with these sensors can be monitored over time to determine a viscosity measurement or other characteristics of the fluid. The fluid input into the canister comes in pulses generated by the variable combination of liquid and gas in the conduit as pulled by the pump or negative pressure source. As the fluid coming into the canister covers and uncovers the first sensor path positioned at a first point within the canister (for example, at the input of the canister) it can create a unique identifier of the fluid coming into the canister over time. As such a ‘barcode’ of how the sensor is covered and/or uncovered by liquid over time at the input to the canister can be a unique identifier. As the fluid runs down the side/drops into the canister (or continues down the fluid flow path into the canister) and contacts the second sensor path at a lower point (or further down the fluid flow path or downstream in the fluid flow path) in the interior of the canister, that unique ‘barcode’ or unique identifier can be time shifted due to the time taken to move under gravity and shifted more (and ‘blurred’) by the viscosity of the fluid. The time offset on the coverage of the sensor can identify the consistency of the fluid. This analysis of the fluid can be used to determine a change in viscosity or change in characteristic of the fluid removed from the wound. This analysis utilizing the two sensors can create a ‘barcode’ or unique identifier of how the sensors are covered and/or uncovered by liquid over time at two points within the canister. This unique identifier determined by the two sensors can be related to the viscosity or other characteristics of the fluid. While this example describes using two sensors to determine the viscosity, change in viscosity, characteristics, or change in characteristics of the fluid within the canister, two or more sensors in multiple arrangements can be monitored over time to determine the viscosity, change in viscosity, characteristics, or change in characteristics of the fluid. To provide this measurement the sensors need to be separated in the fluid path and there can be a change of constraint between the two (e.g. running into a wider volume/dropping under gravity).

Time-based sensing can be used to identify fluid level or characteristics of the fluid within the canister. For example, the identification of movement of the fluid or ‘slosh’ of the fluid can be used to determine a viscosity measurement if the device is moved. The time base of contact with the linear of the canister can be used. The period of the coverage of the sensor with the known dimension of the canister and the gravitational constant gives an indication of the viscosity. The basic ‘slosh’ or movement of the tank is of the form of a natural frequency equation (similar to a pendulum):

$$ω = \sqrt{\frac{g}{l}}$$

where the suspension length (l) is taken as half of the width (w) (in plane) of the canister and g is the gravitational constant.

Applying the depth of the fluid when stationary (d) with the effects of density and viscosity changing the value of the coefficients k and k_{ω} gives the following equation as a possible approximation of the fluid slosh. Depth of the fluid (d) can be similar to the fluid level and determined using any of the approaches described herein.

$$ω = \sqrt{\frac{2 g}{w}} \cdot {k}_{ω} \cdot ( 1 - {e}^{- k \frac{d}{w}} )$$

This can allow identification of viscosity of fluid and change of viscosity of fluid. This allows identification of fluid composition changes of fluid within the canister.

The ratio of covered/uncovered time over a cyclical period combined with the absolute time covered per cycle identifies the level of the fluid in the canister. The position of any given digital sensing path in the canister is a fixed (for example, the fixed sensing devices described herein). The fluid vertical component at any lateral position forms approximately a sinusoidal motion (the amount it varies from a sinusoid depends on the shape of the canister) so the timing at which the fluid creates a contact with the sensor is predictable. The proportion of time that a digital sensor is covered is therefore:

$$\frac{1}{π} {cos}^{- 1} ( \frac{2 Δ}{a} ) between \frac{a}{2} \geq Δ \geq - \frac{a}{2}$$

$$1 where Δ < - \frac{a}{2}$$

$$0 where Δ > \frac{a}{2}$$

Where a is the amplitude of the fluid motion at the lateral position (i.e. approximately zero at the center of the canister and increasing towards the edge) and Δ is the distance of the sensor above the static position of the fluid (i.e. above the fluid is positive and below the fluid is negative). This can generate a partial quantitative value for how full the canister is even from a single pathway. If the device is carried the gait of the patient will provide the inciting oscillation. As such, the sensor can identify increasingly frequent and increasing duration coverage until it is permanently covered.

In some cases, sensors at different levels and/or orientations can be used in the canister. These sensors can allow for a significant increase in granularity both of the fill status and of the disposition of the fluid.

An inertial measurement unit (IMU) can be used to identify the inciting motion for the ‘slosh’ and alter the momentary effective gravity and/or integral of effective gravity for greater granularity of fill sensor read. The IMU can be used for calibration of the sensing device. For example, the sensing system can allow both for the calculation of reaction to a net impulse (i.e. as the IMU is mechanically close-coupled to the body of the pump, the acceleration values measured by the IMU should be almost identical to the values experienced by the fluid-calculation of effect of rotation can incorporate the fixed dimensions of the device and canister). The system can also allow for identification of the components of the impulse to identify compound slosh effects (e.g. a diagonal slosh effect that will not look like a simple harmonic on a single sensing path). The IMU can be used for calibration of any of the sensing devices or sensing techniques described herein. The IMU can be used in combination with any of the sensing devices or sensing techniques described herein for providing information about the patient or canister even outside of calibration uses.

### Shielding of the Fluid Level Sensor

The fluid level sensor 518 can include a mechanical shield 522, as shown in FIG. 12. The mechanical shield 522 can protect the fluid level sensor 518 from splashes and tipping. Beneficially, the mechanical shield 522 can reduce the likelihood of the fluid level sensor 518 triggering a false condition caused by, for example, splashes and/or tipping of the canister 162. The mechanical shield 522 can be disposed inside the canister 162. In some cases, the mechanical shield 522 can be supported by a portion of the canister cap such as, but not limited to, the cap bottom 514 shown in FIG. 5A, the base cap support 1830 shown in FIGS. 5B and 5C, the base support 1650 shown in FIGS. 5D and 5E, or any other component on the inner top surface of the canister. For example, the mechanical shield 522 can be adhered or otherwise attached to a portion of the canister cap or a surface of the interior of the canister. In other cases, the mechanical shield 522 is structurally integral with a portion of the canister cap or a surface of the interior of the canister.

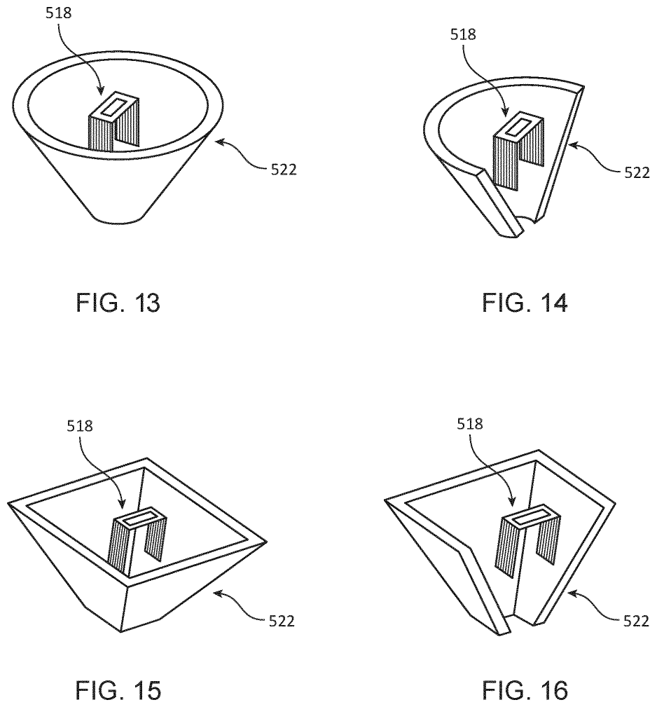

In some cases, the mechanical shield 522 can include an interior surface 522a and an exterior surface 522b and can be configured to surround the fluid level sensor 518, as shown in FIGS. 13-19. In some cases, the mechanical shield 522 comprises an opening along a bottom portion 522c of the mechanical shield 522 and an opening along a top portion 522d of the mechanical shield 522. As used herein, the term bottom can refer to a portion of the canister that is furthest from the canister cap 510 or where the canister 162 connects to the pump assembly, and the term top can refer to a portion of the canister 162 closer to the canister cap 510 or where the canister 162 connects to the pump assembly. The top portion 522d of the mechanical shield 522 can be a portion closer to the canister top or upper surface of the canister than the bottom portion 522c. The mechanical shield 522 can be configured so that the width of the opening along the bottom portion 522c of the mechanical shield 522 is equal to or less than a width of the opening along the top portion 522d of the mechanical shield 522 in at least one plane. The opening along the bottom portion 522c of the mechanical shield 522 can be configured to allow the flow of fluid into an interior portion of the mechanical shield 522 thereby allowing the fluid to contact the arms 520 of the fluid level sensor 518 as the fluid is collecting inside the canister 162. In some cases, the wound fluid can be introduced into the canister 162 via the opening along the top portion 522d of the mechanical shield 522, as shown by arrow 523 in FIG. 20.

FIG. 12 shows a front cross-section view of an example of a mechanical shield 522 for a fluid sensor 518. Beneficially, the mechanical shield 522 of the fluid level sensor 518 can prevent the fluid sensor 518 from falsely triggering a condition when a splash occurs, or the canister 162 is tipped. Although splash-fluid may enter the interior portion of the mechanical shield 522 via the opening along the bottom portion 522c of the mechanical shield 522, the angular profile of the walls of the mechanical shield 522 can allow the splash-fluid to drain along the interior walls of the mechanical shield 522 and exit the interior portion of the mechanical shield 522 via the opening and proceed to collect in a bottom portion of the canister 162 away from the mechanical shield. In some cases, fluid may splash and enter the interior of the mechanical shield and contact one of the two arms 520. However, this splash or contact of one of the two arms will not trigger a canister full or nearly full alarm as both arms are required to be in contact with the fluid to complete the electrical circuit and/or cause a state change in the electrical circuit thereby triggering the canister full or nearly full alarm. In some cases, completing the electrical circuit and/or the state change in the electrical circuit can cause the negative pressure wound therapy device to modify the therapy settings (e.g., increase/decrease a level of negative pressure and/or a fluid flow rate between the negative pressure source, the dressing, and/or the canister).

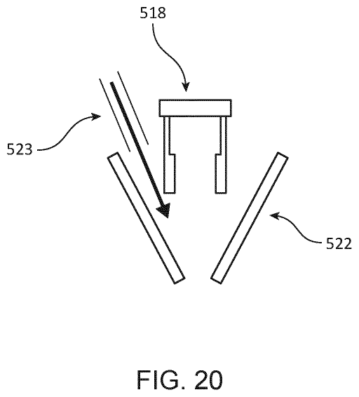

Because the splash-fluid may contact the arms 520 of the fluid sensor 518 as the splash-fluid drains along the interior walls of the mechanical shield 522, the fluid level sensor 518 can be positioned in any configuration that maximizes the distance between the arms 520 and the internal walls of the shield 522, thereby reducing the possibility of the fluid level sensor 518 triggering a false condition. For example, the fluid level sensor 518 shown in FIG. 18 can be positioned as shown in FIG. 19 to increase the distance between the arms 520 of the fluid level sensor 518 and the interior walls of the mechanical shield 522.

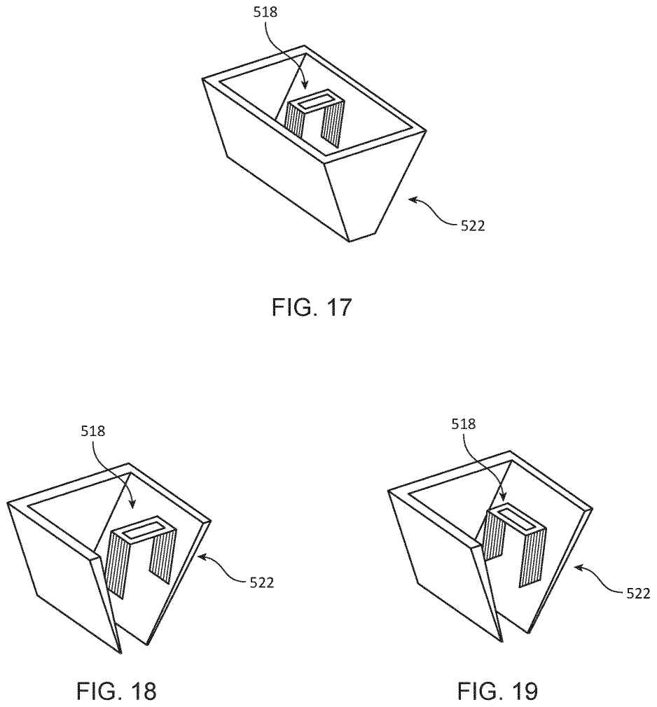

In some cases, the mechanical shield 522 can comprise a conical shape, like that shown in FIGS. 13 and 14. In other cases, the mechanical shield 522 can comprise an inverted truncated square pyramid shape, like that shown in FIGS. 15 and 16. In yet other cases, the mechanical shield 522 can comprise an inverted truncated rectangular pyramid shape, like that shown in FIG. 17. In some cases, the corners of the inverted truncated square pyramid shape and/or the inverted truncated rectangular pyramid shape can include can be curved. Although a conical shape, an inverted truncated square pyramid shape, and an inverted truncated rectangular pyramid shape are disclosed herein as possible shapes of the mechanical shield 522, the mechanical shield 522 can comprise other shapes including non-contiguous and non-symmetrical shapes including a spherical shape, a truncated spherical shape, a hemisphere shape, and/or a truncated hemisphere shape. Further, in some cases, the thickness of the walls of the mechanical shield 522 can be the same across all sections of the mechanical shield 522. In other cases, however, the thickness of the walls may vary across different sections of the mechanical shield 522. In some cases, the mechanical shield can surround the fluid level sensor 518 on all sides as shown in FIGS. 13-19 but can remain open at a top portion and a bottom portion to allow for fluid communication between the fluid level sensor and with the interior of the canister.

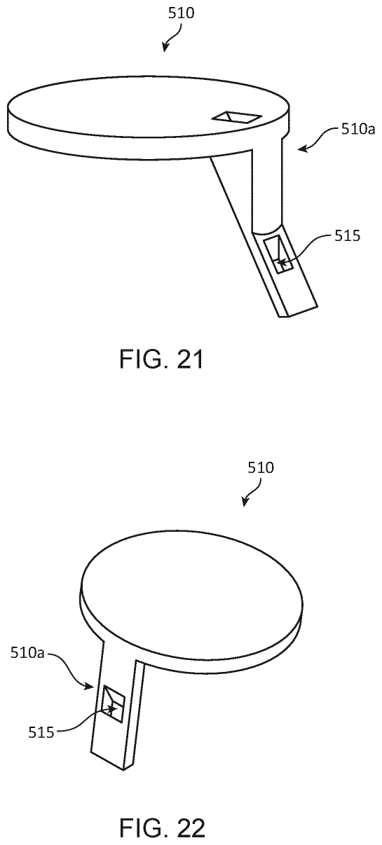

In some cases, as shown in FIGS. 21 and 22, the mechanical shield (similar to mechanical shield 522 shown in FIGS. 15-23) can be defined by a structure surrounding a window 515 positioned on a support surface 510a extending from a cap 510 of the canister. The support surface 510a can extend downwardly from the bottom surface of the canister cap into the interior of the canister. The dimensions and shape of the support structure 510a and the window 515 can vary. For example, and without limitation, the support structure 510a can comprise various widths, lengths, and/or angling relative to the plane of cap 510. Similarly, the size of the window 515 can vary, with some embodiments having narrower windows 515 than others. Further, in some cases, the plane of the window 515 can be parallel or substantially parallel to the plane of cap 510.

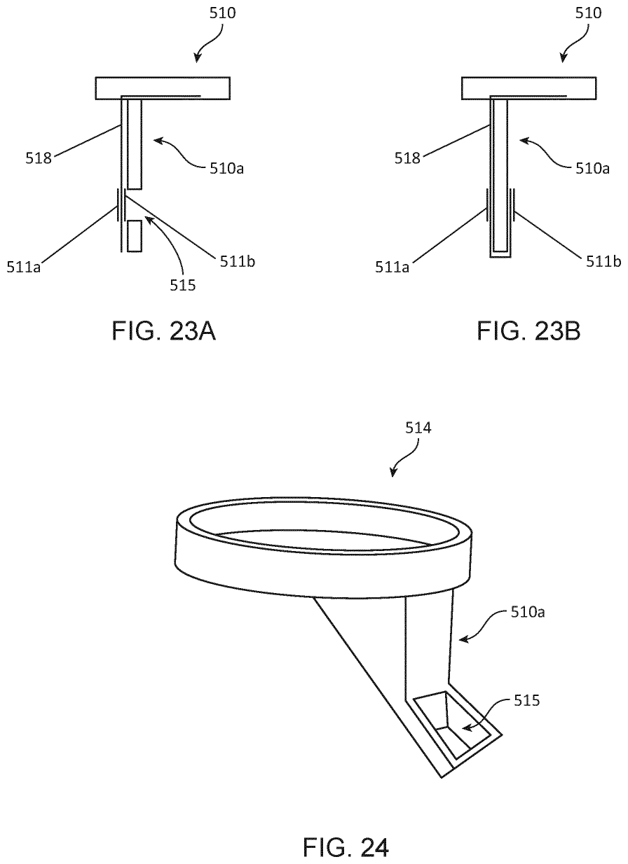

In some cases, the support surface 510a can extend perpendicularly or substantially perpendicularly into the canister interior as shown in FIGS. 23A-23B. As shown in FIG. 23A, the first and second contacts of the fluid level sensor can be placed on one side of the window on the support surface 510a and the contacts would be positioned back to back. In other cases, as shown in FIG. 23B, the first and second contacts of the fluid level sensor can be positioned on different sides of the support surface 510a. FIG. 24 shows another example of a support structure 510a extending from a portion of the canister cap bottom 514 having an opening along a top surface. The opening can define an edge which can facilitate attachment of the portion of the cap bottom 514 to the canister and/or other components of the canister. Even though the canister cap 510 in FIGS. 21-27C is shown in a simplified form, the canister cap 510 can have any of the features from the previous embodiments shown in FIGS. 5A-5E including, without limitation, a central port or structure to communicate with the pump assembly.

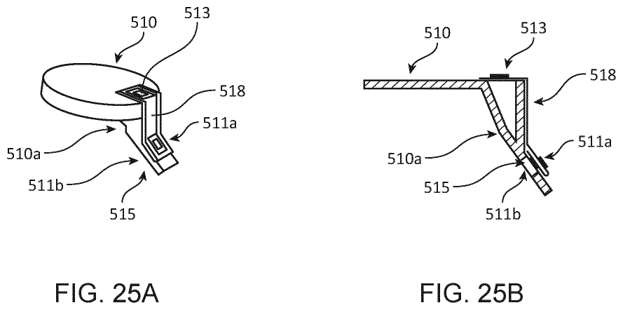

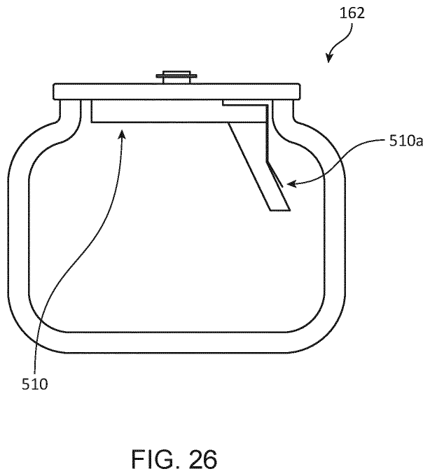

As illustrated in FIGS. 25A-25B, the support surface 510a can extend from the bottom surface of the canister cap and the support surface can extend at an angle relative to the top surface of the canister cap 510. The non-perpendicular orientation relative to the top surface of the cap 514 and the support surface 510a may beneficially result in reduced costs of manufacturing. For example, the example of FIG. 21 can be manufactured with a two-part compression plastic molding tool as opposed to more complicated plastic molding tools. The cap 510 can be disposed inside a top portion of the canister 162, as shown in FIG. 26. The wound fluid within the canister can come in to contact with the fluid level sensor positioned in the window 515 of the support surface 510a. The fluid level sensor can be positioned within the window 515 of the support surface as shown in FIGS. 25A and 25B. For example, as the fluid level within the canister reaches a threshold fluid level, the fluid will come into contact with the fluid level sensor (shown in FIGS. 23A-23B, 25A-25B) within the window 515 of the support surface 510a of the cap 510. As shown in FIGS. 25A and 25B, the first contact surface of the fluid sensor 518 can be positioned on the outward facing surface of the fluid level sensor 518 and the second contact surface can be positioned on the opposite window facing surface of the support surface 510a. The fluid level sensor can then run along the support surface 510a to the top surface of the canister cap 510. An antenna associated with the conductive contact surfaces of the fluid level sensor can be positioned on the top surface of the canister cap 510 as shown in FIGS. 25A and 25B.

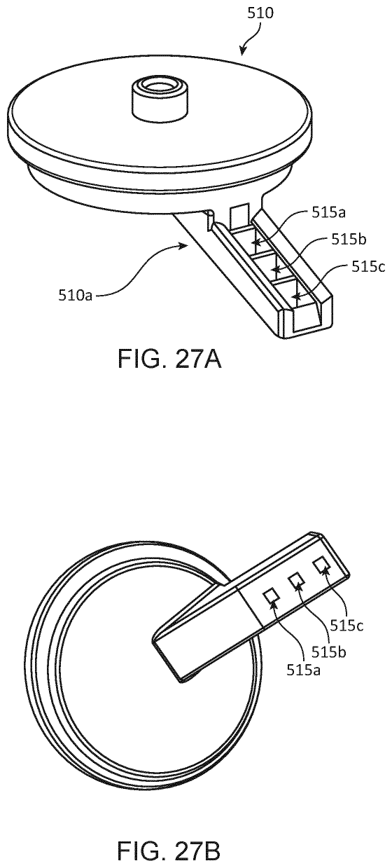

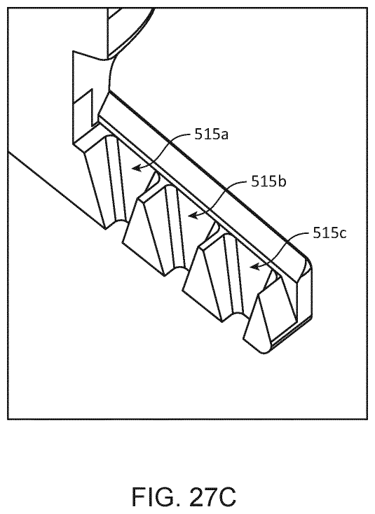

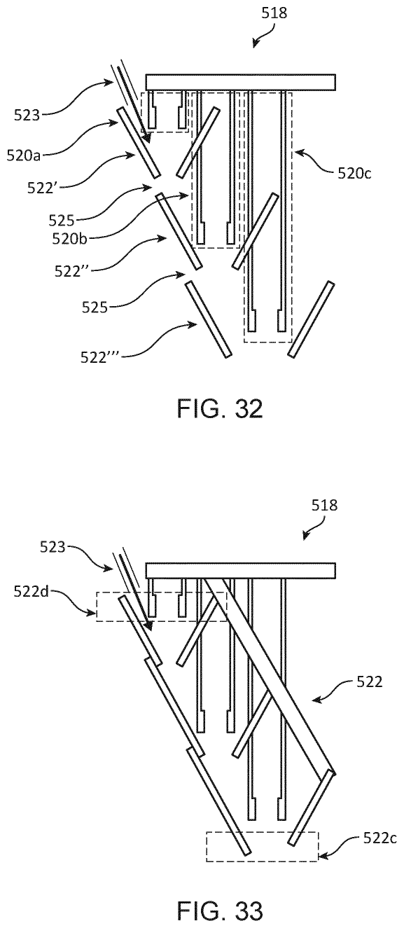

In some cases, a support surface 510a of the cap 510 can include one or more windows 515a, 515b, and 515c along the support surface 510a of the cap 510 each defining an area of a mechanical shield, as shown in FIGS. 27A-27C. For example, in some cases, the one or more areas of the one or more windows 515a, 515b, and 515c can correspond to the areas shown in FIG. 32, 522′, 522″, 522″″ of the mechanical shield 522 which is discussed in more detail herein. The area of the one or more openings along a bottom surface of the support surface 510a associated to the one or more windows 515a, 515b, and 515c can vary. For example, similar to the varying sizes of the mechanical shield structures described herein, the size of the opening associated to window 515a can be smaller than the size of the opening associated to window 515b, and the size of the of the opening associated to window 515b can be smaller than the size of the opening associated to window 515c, as shown in FIG. 27B. Alternatively, the size of the opening associated to window 515a can be larger than the size of the opening associated to window 515b, and the size of the of the opening associated to window 515b can be larger than the size of the opening associated to window 515c. The varying sizes can allow for detection or determination of characteristics of the fluid as described herein. As shown in FIG. 27C, the one or more windows 515a, 515b, and 515c can be positioned at different elevations. For example, window 515a can be at an elevation closer to cap 510 than window 515b and window 515c. As explained in further detail below, these configurations can beneficially allow one or more fluid level sensors 518, and/or or a single fluid level sensor 518 having one or more sets of arms 520, to provide an indication of the composition of the wound exudate, the amount of fluid collected inside the canister 162, the rate at which fluid collects inside the canister 162, a viscosity of the wound exudate, and/or a faulty condition of the negative pressure wound treatment system.

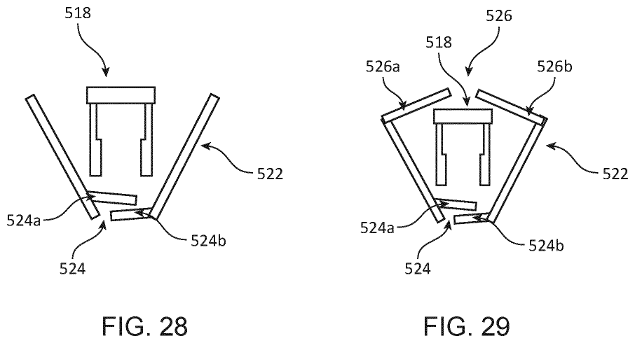

In some case a sensor, such as the sensor 518 shown in FIGS. 25A-25B, can be positioned within one or more of the windows 515a-515c. The position of the fluid level sensors 518 (or paths) within the windows 515a-515c, can allow the fluid level sensors 518 to provide an indication of a viscosity of the wound exudate. For instance, as the fluid coming into the canister covers and uncovers a first fluid level sensor 518 (or path) positioned at the first window 515a, it can create a unique identifier of the fluid coming into the canister over time. As such a ‘barcode’ of how the sensor is covered and/or uncovered by liquid over time at the input to the canister can be a unique identifier. As the fluid runs down the side/drops into the canister (or continues down the fluid flow path into the canister) and contacts a second fluid level sensor 518 in the second window 515b, that unique ‘barcode’ or unique identifier can be time shifted due to the time taken to move under gravity and shifted more (and ‘blurred’) by the viscosity of the fluid. The time offset on the coverage of the sensor can identify the consistency of the fluid. This analysis of the fluid can be used to determine a change in viscosity or change in characteristic of the fluid removed from the wound. In some cases, the mechanical shield 522 includes a splashguard element, as shown in FIGS. 28 and 29. The splashguard element can include a first set of guards 524 comprising one or more guards configured to prevent splash-fluid from entering the interior portion of the mechanical shield 522 through the opening along the bottom portion 522c (shown in FIG. 12) of the mechanical shield 522. In some cases, the splashguard element can include one or more guards positioned substantially along the bottom portion 522c of the mechanical shield 522. For example, the splashguard can include a first guard 524a extending from a first portion of the interior surface 522a of the mechanical shield 522, and a second guard 524b extending from a second portion of the interior surface 522a of the mechanical shield 522. In some cases, the second portion of the interior surface 522a (shown in FIG. 12) of the mechanical shield 522 is a portion opposite the first portion. At least a portion of the first guard 524a can overlap a portion of the second guard 524b. In other cases, the first guard 524a does not overlap with the second guard 524b. The first set of guards 524 can be arranged to create a fluid pathway from the exterior of the mechanical shield 522 to the interior of the mechanical shield 522. Beneficially, the fluid pathway can facilitate the flow of fluid from the interior of the canister 162 to an interior portion of the mechanical shield 522 as the canister 162 fills, while preventing splash-fluid from triggering the fluid level sensor 518.

In yet other cases, the splashguard element includes a second set of guards 526 comprising one or more guards positioned substantially along a top portion 522d (shown in FIG. 12) of the mechanical shield 522, as shown in FIG. 29. The second set of guards 526 can prevent splash-fluid from entering an interior portion of the mechanical shield 522 through a top portion 522d of the mechanical shield 522 and contacting the arms 520 of the fluid sensor 518, thereby reducing the possibility of the fluid sensor 518 triggering a false condition. In some cases, the second set of guards 526 can include a first guard 526a extending from a first portion of the interior surface 522a of the mechanical shield 522, and a second guard 526b extending from a second portion of the interior surface 522a of the mechanical shield 522. Similar to the first guard 524a of the first set of guards 524, at least a portion of the first guard 526a can overlap a portion of the second guard 526b. In other cases, the first guard 526a does not overlap with the second guard 526b. The second set of guards 526 can be arranged to create a fluid pathway from the exterior of the mechanical shield 522 to the interior of the mechanical shield 522. In other cases, the splashguard element comprises a single guard with an opening. The single guard can be positioned/shaped to cover at least a top portion of the fluid sensor 518.

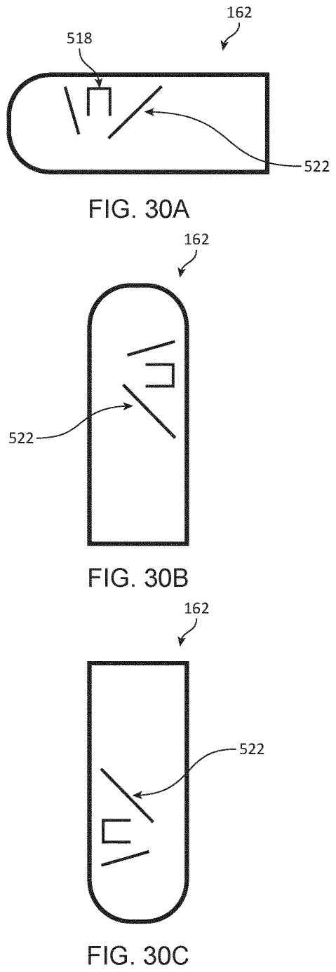

The canister 162 of the negative pressure wound therapy device can be shaped so that the canister 162 can sit in one or more stable positions. The fluid sensor 518 can be positioned to detect a condition (e.g., canister full condition) notwithstanding the stable position in which the canister 162 sits. The mechanical shield 522 disclosed herein can be positioned to protect the fluid level sensor 518 from splashes and/or tipping of the canister 162 notwithstanding the stable position in which the canister 162 sits, as shown in FIGS. 30A-30C. FIGS. 30A and 30B show a cross-section of a canister 162 in a first stable position and a second stable position respectively. When the canister 162 is in the first or second stable position, the mechanical shield 522 can protect the fluid level sensor 518 from splashes. Beneficially, the angle of the walls of the mechanical shield 522 when the canister 162 is in the first or second stable positions allows fluid inside the mechanical shield 522 to drain and continue collecting inside the canister 162. When the canister 162 is not in a stable position, like that shown in FIG. 30C, the fluid level sensor 518 may fail to detect a condition inside the canister 162 (e.g., canister full condition). The angle of the walls of the mechanical shield 522 may lead at least a portion of the fluid inside the mechanical shield 522 in the direction of the filter 516 instead of leading the fluid to a bottom portion of the canister 162. This may cause the filter 518 to block and/or fluid to leak, both of which can result in loss of therapy, before the fluid level sensor 518 detects a condition inside the canister 162.

### Fluid Level Sensor Arrangement

In some cases of the negative pressure wound therapy device, an NFC tag having a first end and a second end opposite the first end can be disposed inside the canister 162. The first end of the NFC tag can be secured to an inner base of the canister 162. The chip and/or antenna of the NFC tag can be positioned along the second end of the NFC tag. The second end of the NFC tag can be configured to float as fluid starts collecting inside the canister 162. As more fluid collects inside the canister 162, the second end of the NFC tag will float higher relative to the base of the canister 162. The NFC tag can be configured to communicate with an NFC reader located within the negative pressure wound therapy device once the second end of the NFC tag reaches a threshold fluid level within the canister 162 (e.g., once the fluid inside the canister 162 reaches a threshold level). The communication from the NFC tag to the NFC reader can indicate a condition inside the canister 162 (e.g., canister full or nearly full condition).

The negative pressure wound therapy device can include a guide disposed inside the canister 162. The guide can extend from a bottom portion of the canister 162 to a top portion of the canister 162 and be configured to at least partially surround the NFC tag. In some cases, the guide can be formed along an interior wall of the canister 162. Beneficially, the guide can guide the second end of the NFC tag from a bottom portion of the canister 162 to a known location on a top portion of the canister 162 and prevent the second end of the NFC tag from floating freely inside the canister 162 as fluid starts collecting.

The negative pressure wound therapy device can include one or more NFC tags disposed inside the canister 162. Each NFC tag of the one or more NFC tags and the NFC reader can be configured to communicate based at least in part on each NFC tag reaching a threshold fluid level within the canister 162. Further, each NFC tag of the one or more NFC tags can be of a different density thereby allowing each NFC tag to float as fluid collects inside the canister 162 if the density of the fluid inside the canister 162 is higher than the density of each individual NFC tag. Beneficially, the one or more NFC tags can provide an indication of the composition of the fluid collected within the canister 162.

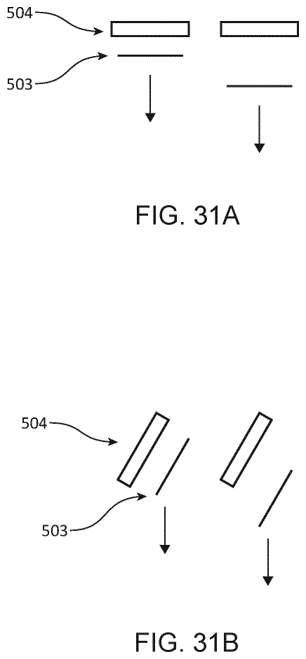

In some cases, the NFC tag antenna 503 and NFC reader 504 are positioned perpendicular to a removal direction ((e.g., the direction in which canister 162 is removed from the pump assembly 110) of the canister 162, as shown in FIG. 31A. In this configuration, the NFC tag antenna 503 and NFC reader 504 can continue to communicate even when the distance between the NFC antenna 403 and NFC reader 504 increases. This can occur if the canister 162 is tipped or moved. In some cases, the NFC tag antenna 503 and NFC reader 504 are positioned non-perpendicular to the removal direction of the canister 162, as shown in FIG. 31B. A non-perpendicular configuration can result in the NFC tag antenna 503 and the NFC reader 504 losing signal if the distance between the NFC tag antenna 503 and NFC reader 504 increases (or at least losing signal at a shorter distance than the distance necessary to lose signal with a perpendicular arrangement as shown in FIG. 31A). Beneficially, in the non-perpendicular configuration as illustrated in FIG. 32, the loss of signal at a shorter distance can allow for a more sensitive sensor configuration. This would allow for a strong signal when the antenna and reader are coupled together properly and a quicker drop off in signal when the canister and pump assembly are separated. This can reduce the acceptance of an unstable condition and would make the system more sensitive to detecting any looseness in the coupling of the components that can lead to leaks.

The negative pressure wound therapy device can include one or more fluid level sensors 518, as shown in FIGS. 32-35. The negative pressure wound therapy device can also include a single fluid level sensor 518 having one or more sets of arms 520, as shown in FIGS. 32 and 33. For example, a fluid level sensor 518 can include a first set of arms 520a, a second set of arms 520b, and a third set of arms 520c, as shown in FIG. 32. The length of the first 520a, second 520b, and third 520c sets of arms can vary. The first set of arms 520a can be shorter than the second set of arms 520b, and the second set of arms of 520b can be shorter than the third set of arms 520c. However, other combinations are possible. The different lengths of the first 520a, second 520b, and third 520c sets of arms can allow the fluid level sensor 518 to detect one or more conditions inside the canister 162 as fluid collects inside the canister 162.

In some cases, the one or more fluid level sensors 518, or the one or more sets of arms 520 of a single fluid level sensor 518, can be positioned at different elevations relative to the canister cap 510. This means that some fluid levels sensors 518 can be closer to the canister cap 510 than other fluid level sensors 518. Beneficially, the use of the one or more fluid level sensors 518, or the one or more sets of arms 520 of a single fluid level sensor 518, at different vertical positions (e.g., elevations) can allow for a rate of inflow measurement calculation. Fluid level sensors 518 can be configured so that fluid level sensors 518 positioned farther away from the canister cap 510 trigger before fluid level sensors 518 positioned closer to the canister cap 510 as the canister fills with fluid. A rate of inflow measurement can be obtained by measuring the time it takes for each fluid level sensor 518 to trigger. The triggering of an out-of-sequence fluid level sensor 518 can indicate a failure of one or more of the fluid level sensors 518. For example, the triggering of a first fluid level sensor 518 prior to the triggering of a second fluid level sensor located farther away from the canister cap 510 than the first fluid level sensor 518 can indicate that the first and/or second fluid level sensors 518 are faulty. Because the negative pressure wound therapy device may remove fluid from the wound at a higher rate when therapy begins and at a lower rate as therapy progresses, it may be desirable for the spacing between the one or more fluid level sensors 518 to vary. That is, the one or more fluid level sensors 518, and/or the fluid level sensor 518 having one or more sets of arms 520, can be positioned at different elevations, with fluid level sensors 518 closer to each other at a top portion of the canister 162 than at a bottom portion of the canister 162. Beneficially, this may provide an indication of whether the wound is following an expected progress (e.g., whether a particular fluid level sensor 518 triggered before expected; meaning the fluid rate is higher than expected).

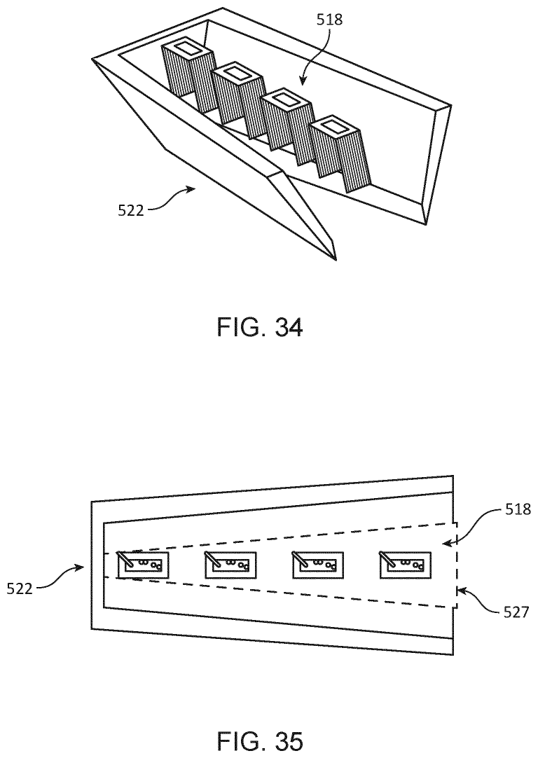

In other cases, the one or more fluid level sensors 518, or the one or more sets of arms 520 of a single fluid level sensor 518, can be positioned at different lateral positions relative to the walls of the canister 162 but at the same elevation relative to the canister cap 510, as shown in FIG. 34. The one or more fluid level sensors 518 can be configured to trigger at the same time. Beneficially, the failure of at least one of the fluid level sensors 518 would indicate that at least one of the one or more fluid level sensors 518 is faulty.

With respect to FIGS. 27A-27C, and 34-35, and as described in relation to FIGS. 27A-27C, the position of the sensors 518 within the mechanical shield 522 can beneficially allow one or more of the sensors 518, and/or or a single sensor 518 having one or more sets of arms 520, to provide an indication of the composition of the wound exudate, the amount of fluid collected inside the canister 162, the rate at which fluid collects inside the canister 162, a viscosity of the wound exudate, and/or a faulty condition of the negative pressure wound treatment system.

The position of the fluid level sensors 518 (or paths) within the mechanical shield 522 can allow the fluid level sensors 518 to provide an indication of a viscosity of the wound exudate. For instance, as the fluid coming into the canister contacts a first fluid level sensor 518 (or path), it can create a unique identifier of the fluid coming into the canister over time. As such a ‘barcode’ of how the sensor is covered and/or uncovered by liquid over time at the input to the canister can be a unique identifier. As the fluid runs down the side/drops into the canister (or continues down the fluid flow path into the canister) and contacts a second fluid level sensor 518, that unique ‘barcode’ or unique identifier can be time shifted due to the time taken to move under gravity and shifted more (and ‘blurred’) by the viscosity of the fluid. The time offset on the coverage of the sensor can identify the consistency of the fluid. This analysis of the fluid can be used to determine

In some cases, the one or more fluid level sensors 518, or the one or more sets of arms 520 of a single fluid level sensor 518, can include a mechanical shield 522, as shown in FIGS. 20, 32-35. For example, each set of arms of the fluid level sensor 518 can be associated to at least one area of the mechanical shield. As shown in FIG. 32, the first set of arms 520a can be protected by a first area 522′ of the mechanical shield 522, the second set of arms 520b can be protected by a second area 522″ of the mechanical shield 522, and the third set of arms 520c can be protected by a third area 522′″ of the mechanical shield 522. The mechanical shield 522 can comprise different shapes and/or sizes including non-contiguous and non-symmetrical shapes and any shape or size described for the other examples of mechanical shields described herein.

The width of the openings along a bottom portion and a top portion of each area of the mechanical shield 522 can vary. For example, the width of the openings along the top portion and the bottom portion of the first area 522′ of the mechanical shield 522 can be smaller than the openings along the top portion and the bottom portion of the second area 522″ of the mechanical shield 522. Similarly, the width of the openings along the top portion and the bottom portion of the second area 522″ of the mechanical shield 522 can be smaller than the openings along the top portion and the bottom portion of the third area 522′″ of the mechanical shield 522. In cases where the width of the openings along the top portion and the bottom portion of the first area 522′ of the mechanical shield 522 are smaller than the openings along the top portion and the bottom portion of the second and third areas 522″, 522′″ of the mechanical shield 522, thin fluid can drain along the interior surfaces of the different mechanicals shields areas 522′, 522″, 522′″ without triggering any of the sets of arms of 520a, 520b, 520c of the fluid level sensor 518. If, however, the fluid is thick, then the fluid may start collecting inside the first area 522′ of the mechanical shield 522 thereby triggering the first set of arms 520a of the fluid level sensor 518. At least a portion of the overflow from the first area 522′ can flow to the next area of the mechanical shield 522, that is, area 522″. Depending on the thickness of the fluid, the overflow can fill the second area 522″ thereby triggering the second set of arms 520b or drain along the interior surface of area 522″ without triggering the second set of arms 520b. The thickness of the fluid inside the canister can be measured by the order in which the one or more sets of arms 520a, 520b, 520c trigger. Beneficially, this can provide an indication of the composition of the fluid collected inside the canister.

The mechanical shield 522 can also be configured so that the width of the openings along the top portion and the bottom portion of the first area 522′ of the mechanical shield 522 are larger than the openings along the top portion and the bottom portion of the second area 522″ of the mechanical shield 522. Similarly, the width of the openings along the top portion and the bottom portion of the second area 522″ of the mechanical shield 522 can be larger than the openings along the top portion and the bottom portion of the third area 522′″ of the mechanical shield 522. In this case, fluid would drain along the interior surfaces of the one or more areas 522′, 522′ 522′″ of the mechanical shield 522 until and unless the fluid starts filling one of the one or more areas of the mechanical shield 522′, 522″ 522′″. The mechanical shield can be configured so that the overflow from one of the areas 522′, 522′ 522′″ continues to collect in the canister 162. Drained fluid from the one or more areas 522′, 522′ 522′″ can flow to the area below if there's one, or to the canister 162. The size of the opening along the bottom portion of the one or more areas 522′, 522″ 522′″ of the mechanical shield 522 can affect how fluid collects or drains to the next area. For example, fluid can collect faster in areas 522′, 522′ 522′″ having smaller openings than it does in areas 522′, 522′ 522′″ having bigger openings. The thickness of the fluid inside the canister can be measured by the order in which the one or more sets of arms 520a, 520b, 520c trigger. Beneficially, this can provide an indication of the composition of the fluid collected inside the canister. Although the mechanical shield 522 in FIGS. 32 and 33 shows a mechanical shield having three mechanical shield areas 522′, 522″, and 522′″, the mechanical shield 522 can include more than three mechanical shield areas or less than three mechanical shield areas.

In some cases, the mechanical shield 522 can include an opening along a top portion 522d of the mechanical shield and an opening along a bottom portion 522c of the mechanical shield, as shown in FIG. 33. In these cases, the wound fluid can enter the mechanical shield 522 through the opening along the top portion 522d of the mechanical shield 522, as shown by arrow 523, drain and exit the mechanical shield 522 via the opening along the bottom portion 522c of the mechanical shield 522, and/or spill (e.g., overflow) and exit the mechanical shield 522 via the opening along the top portion 522d of the mechanical shield 522. In this configuration, the fluid overflowing from the opening along the top portion 522d of the mechanical shield 522 or draining from the opening along the bottom portion 522c of the mechanical shield 522 can continue to collect in the canister 162. In some cases, the mechanical shield 522 shown in FIG. 33 can also include at least one opening, like opening 525 in FIG. 32, located between the top portion 522d of the mechanical shield 522 and the bottom portion 522c of the mechanical shield 522. The at least one opening can provide an additional path for the fluid inside the mechanical shield 522 to spill (e.g., overflow) and exit the mechanical shield 522 and continue to collect in the canister 162

In some cases, the mechanical shield 522 can comprise one or more openings 525 along two areas of the of the mechanical shield 522, as shown in FIG. 32. For example, the one or more openings 525, can be located between the first area 522′ of the mechanical shield 522 and the second area 522″ of the mechanical shield 522, as shown in FIG. 32. The one or more openings can also be located between the second area 522″ of the mechanical shield 522 and the third area 522′″ of the mechanical shield 522, as shown in FIG. 32. In cases where the mechanical shield 522 includes one or more openings 525, the wound fluid can drain and exit each area (e.g., 522′, 522″, 522′″) of the mechanical shield 522 and/or spill (e.g., overflow) and exit each area (e.g., 522′, 522″, 522′″) of the mechanical shield 522 through the one or more openings 525. In cases having one or more openings 525 between each area of the mechanical shield 522, at least a portion of the fluid overflowing from each area of the mechanical shield 522 can continue to collect in the canister 162, and at least a portion of the fluid drained from each area of the mechanical shield 522 can continue to collect in the area of the mechanical shield 522 below, if there is one, or in the canister 162. Beneficially, the angular profile of the walls of the mechanical shield 522 can allow any fluid entering an interior portion of the mechanical shield 522 to drain along the interior walls of the mechanical shield 522.

The mechanical shields 522 can provide an indication of the composition of the fluid collected inside the canister 162. For example, in some cases the fluid can be introduced into the mechanical shield 522 through a top portion of the mechanical shield 522, as shown by arrow 523 in FIGS. 32 and 33. Depending on the surface tension and density of the fluid collected, any fluid entering an interior portion of the mechanical shield 522 can drain out of each area of the mechanical shield 522 or fill the interior of each area of the mechanical shield 522 thereby triggering the fluid level sensor 518 corresponding to that area of the mechanical shield 522. By identifying which areas of the mechanical shield 522 drain and which areas of the mechanical shield 522 fill, the composition of the fluid inside the one or more areas of the mechanical shield 522 can be identified. Beneficially, this can provide an indication of the status of the wound, for example, the density of the fluid can indicate if the wound exudate has too much blood or other characteristics that can indicate the negative pressure wound therapy may need to be paused or discontinued. Additionally, the triggering of the same one or more of the fluid level sensors 518 can also operate to indicate a canister full condition.

In some cases, a single mechanical shield 522, having any of the shapes, dimensions, or characteristics of the mechanical shields disclosed herein, and having an aperture along an upper portion of the mechanical shield 522 and an aperture along a bottom portion 522c of the mechanical shield 522 can protect one or more fluid level sensors 518, and/or a fluid level sensor 518 having one or more sets of arms 520. In this configuration, the one or more fluid level sensors 518, and/or a fluid level sensor 518 having one or more sets of arms 520, can be positioned at different elevations above the opening along the bottom portion 522c of the mechanical shield 522. The pressure of the fluid inside the mechanical shield 522 relative to the surface tension of the interior walls of the mechanical shield 522 can create a level of fluid in the mechanical shield 522. The level of fluid inside the mechanical shield 522 can be detected by the one or more fluid level sensors 518, and/or a fluid level sensor 518 having one or more sets of arms 520. The triggering of the one or more fluid level sensors 518, and/or the fluid level sensor 518 having one or more sets of arms 520 can also operate to indicate a canister full condition. In some cases, the fluid level sensors can be positioned within a cylindrical mechanical shield with one or more circular cross-section holes at the bottom.

In some cases, the mechanical shield 522 can include an opening of varying cross-section along a bottom portion of the mechanical shield 522, as shown in FIGS. 34-35. The opening along the bottom portion of the mechanical shield 522 can define a channel 527, as shown in FIG. 35, along an interior portion of the mechanical shield 522. Fluid can be introduced into the canister 162 via the opening along a top portion of the mechanical shield 522 at the end of the smallest cross-section. Properties of the fluid, such as its density and/or surface tension, can be defined by how many of the one or more fluid level sensors 518 trigger as the fluid flows through the channel 527. In some cases, one end of the arms 520 of the one or more fluid level sensors 518 can be positioned above the channel 527 so that the one or more fluid level sensors 518 trigger as the fluid fills an interior portion of the mechanical shield 522. In some cases, the one or more fluid level sensors 518 are positioned below the channel 527 of the mechanical shield 522. In this configuration, a single fluid level sensor 518 of the one or more fluid level sensors 518 would trigger at a time, representing the point along the channel 527 where the fluid has dripped and contacted the single fluid level sensor 518.

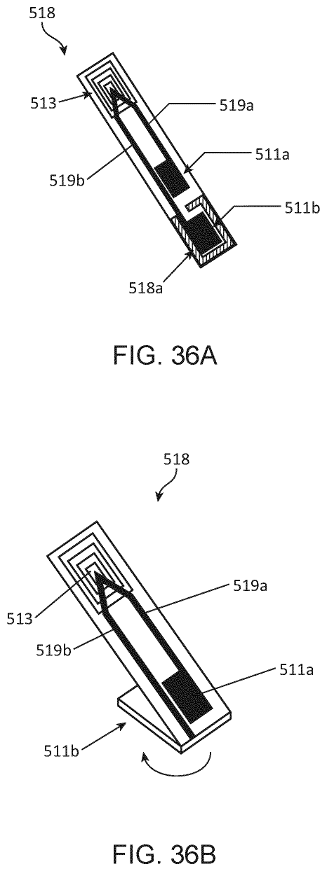

FIGS. 36A and 36B illustrate examples of a fluid level sensor 518. In some cases, the fluid level sensor 518 shown in FIGS. 36A and 36B can be similar to the fluid level sensor 518 shown in FIGS. 25A and 25B. The fluid sensor 518 can include an antenna and at least two contact points (pad 1 and pad 2) that can trigger the sensor when both are in contact with fluid. In some cases, the contact points (pad 1 and pad 2) can have conductive material. The contacts can be in electrical communication with the antenna through their individual tracks as shown in FIGS. 36A and 36B. In some cases, the individuals tracks shown in FIGS. 36A and 36B can be coated with a non-conductive material. This can prevent fluid from triggering the fluid level sensor 518 if fluid contacts a portion of the fluid level sensor 518 where the individual tracks are located. In some cases, the fluid level sensor 518 can also include an adhesive surface along at least a portion of the fluid level sensor 518. The adhesive portion can be located, for example, at or near at least one of the two contact points, as shown in FIG. 36A. The underside of the at least one of the two contact points can also include an adhesive surface. The adhesive portion can facilitate attachment of the fluid level sensor to, for example, and without limitation, an interior portion of the canister or one or more of the components disposed inside the canister, as shown in FIGS. 25A and 25B. FIG. 36B illustrates the fluid sensor 318 with the first contact (pad 1) folded behind the sensor and therefore not visible in FIG. 36B.

In some cases, sealing a sensor, such as fluid level sensor 518, into a canister, such as canister 162, can complete a normally closed circuit, for example a normally closed circuit such as at least one of a tamper detection system (e.g., the tamper detection system, shown in FIGS. 6A and 6B), and the fluid level sensor 518. Successful closure of the tamper detection system and/or the fluid level sensor can provide an indication that the assembly of the canister is correct.

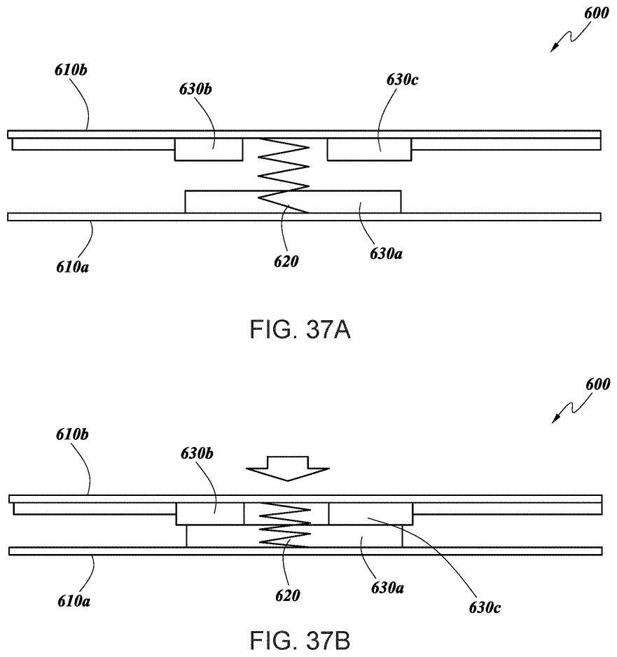

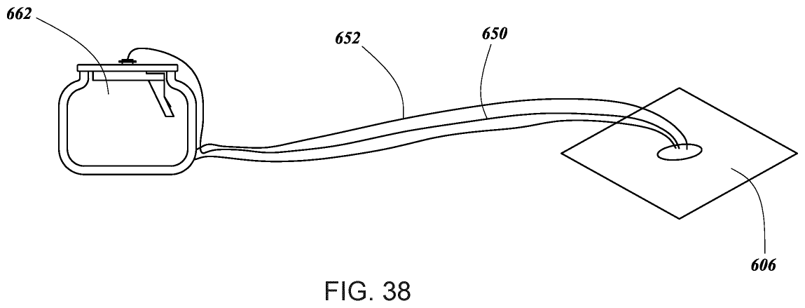

Any of the wound therapy devices described herein can include a sensor path extending from the dressing to the canister as shown in FIG. 38. As shown in FIGS. 37A-37B, the sensor path 600 can include a first membrane 610a, a second membrane 610b, at least one spring 620 (and/or elastomeric spacer), and at least one conductive path. The first and second membranes 610a, 610b can be airtight (e.g., sealing the sensor path 600). In some cases, the sensor path can include three conductive paths 630a, 630b, 630c. A first conductive path 630a can be positioned on the first membrane 610a while a second conductive path 630b and a third conductive path 630c can be positioned on the second membrane 610b. In some cases, the sensor path 600 can include a normally-open configuration. That is, the sensor path 600 can be configured so that the first conductive path 630a is not in contact with the second and third conductive paths 630b, 630c when negative pressure treatment has not been applied, negative pressure treatment has been paused, or negative pressure treatment has not reached a threshold limit. In some cases, the threshold limit can represent the point at which, depending on the duration of a negative pressure treatment session, the system to which the sensor path 600 is associated to is expected to reach a condition (e.g., negative pressure threshold is reached). When negative pressure treatment is applied, or the negative pressure treatment reaches a threshold limit, the spring 620 between the first and second membranes 610a, 610b can compress and cause the first, second, and third conductive paths 630a, 630b, 630c to contact each other, as shown in FIG. 37B. The first, second, and third conductive paths 630a, 630b, 630c can complete a circuit and/or there cause a state change in the electrical circuit when they are in contact with each other. The complete circuit can provide an indication about the condition of the system (e.g., the presence of negative pressure and/or that a negative pressure threshold has been reached). In some cases, discontinuing negative pressure wound therapy can cause the spring 620 to decompress. This can cause the first, second, and third conductive paths 630a, 630b, 630c to stop contacting each other and break the completed circuit.

As shown in FIG. 38, the sensor path 600 can extend from a portion of a dressing 606 to a canister 662. The sensor path 600 can beneficially identify the presence of negative pressure at the dressing 606 and/or a port thereof. For example, the sensor path 600 can be located within a soft port of the dressing 606 and/or elsewhere in the dressing 606 and can be used to identify/sense the presence of negative pressure therein. The sensor path 600 can obtain a negative pressure reading that is detected by the canister 662 and the negative pressure reading can be communicated to a related pump assembly (for example, pump assembly 160, as shown in FIG. 2A) through any communication between the pump assembly and canister assembly described herein or any known communication methods. In some cases, the sensor path 600 can include one or more connections and/or one or more materials. For example, the sensor path 600 can include a dual-core or co-axial cable. In some cases, the cable 650 can extend parallel to a vacuum tube 652 extending from the dressing 606 to the canister 662 or inside the vacuum tube 652, as shown in FIG. 38. Any of the negative pressure wound treatment sensors described herein can be positioned inside a flexible conduit used with the dressing or any location inside the dressing.

In some cases, the arms, for example arms 520 (see FIGS. 7-9), of any of the fluid level sensors disclosed herein, including but not limited to fluid level sensor 518, can include a coating. In some cases, the coating can be dissolvable and/or nonconductive. In some cases, when the dissolvable material is conductive, the dissolvable material can form a portion of a circuit formed by the arms 520 of the fluid level sensor and the dissolvable material. When the dissolvable material dissolves, the gap between the arms 520 where the dissolvable material was located before dissolving, can create high resistance in the circuit thereby causing the signal of the circuit to change. The changing signal can cause the fluid level sensor to detect a condition inside the canister (e.g., canister full condition). In some cases, when the dissolvable material is conductive, the dissolvable material can act as a bracing element separating the arms 520 of the fluid level sensor 518. When the dissolvable material dissolves, at least one isolating element, such as an air gap, can cause the circuit created by the arms 520 and the dissolvable material to break thereby causing the signal of the circuit to change. The changing signal can cause the fluid level sensor to detect a condition inside the canister (e.g., canister full condition). The coating can beneficially prevent the fluid level sensor from immediately detecting a condition inside the canister when the arms 520 momentarily contact the fluid within the canister. For example, the coating can dissolve after being in contact with a fluid inside the container for a minimum period of time (e.g., 1 minute, 2 minutes, 3 minutes, 4 minutes, etc.).

In some cases, one or more of the arms 520 can include a coating. The coating on the arms 520 can dissolve when the coating is contact with the fluid aspirated from the wound. In some cases, only one of the arms 520 includes a coating. However, in fluid level sensors 518 including more than one arm 520, all arms 520 can include a coating. In some cases, a physical property of the coating can change when the coating contacts the fluid collected from the wound. The change in physical property can cause the fluid level sensor to detect a condition inside the canister (e.g., canister full condition). In some cases the physical property includes conductivity.

In some cases, only continuous exposure to the fluid inside the canister will cause the coating to dissolve. After the coating is dissolved, the arms of the of the fluid level sensor can contact the fluid directly thereby completing the circuit and allowing the fluid level sensor to detect a condition inside the canister (e.g., canister full condition). Before the coating of the arms is fully dissolved, fluid contacting the arms of the fluid level sensor will not cause the fluid level sensor to detect a condition inside the canister. Beneficially, this can prevent the fluid level sensor from erroneously detecting a condition inside the canister when, for example, fluid splashes or droplets momentarily contact the arms of the fluid level sensor. In some cases, the coating can include a dissolvable non-conductive material. In some cases, the coating can include a Poly Vinyl Alcohol, a water based Poly Vinyl Alcohol, a Poly Vinyl Acetate, silicon oxides, and/or silicon nitrides, which can have different or similar dissolution properties (e.g., time to dissolve, ability to fully dissolve and/or disperse, etc.) In some cases, the arms of the fluid level sensor can include a film. The PVA can be added to the arms of the fluid level sensor by coating, bonding a film, or deposition. In some cases, the coating on each arm can occupy at least a portion of the gap between the arms.

In some cases, the arms of the fluid level sensor can also include a hydrophobic layer and/or an oleophobic layer. The hydrophobic and/or oleophobic layer can be in addition to the dissolvable coating. The hydrophobic and/or oleophobic layer can prevent fluid splash and fluid droplets from sitting on the arms of the fluid level sensor. The hydrophobic and/or oleophobic layer can cause the fluid splash and fluid droplets to drain into the canister. This can beneficially prevent the fluid splash and fluid droplets from prematurely dissolving the coating on the arms and thus prevent the fluid level sensor from erroneously detecting a condition inside the canister. In some cases, continuous exposure to the fluid inside the canister by the arms for a minimum period of time will cause the coating to dissolve notwithstanding the hydrophobic and/or oleophobic layer. For example, when the fluid inside the canister reaches a level where it contacts both arms of the fluid level sensor, constant exposure to the fluid by the arms will cause the coating to dissolve. This can cause the fluid to directly contact the arms of the fluid level circuit and complete the circuit. A complete circuit can cause the fluid level sensor to detect a condition inside the canister (e.g., canister full condition).

Any of the fluid level sensors disclosed herein can include a dissolvable material. The dissolvable material can include, for example, silicon oxides and/or silicon nitrides. In some cases, the dissolvable material can be positioned between the arms 520 of any of the fluid level sensor 518. In some cases, the dissolvable material can create a circuit with the arms 520 when the canister is not full. The dissolvable material can dissolve as fluid starts collecting inside the canister. For example, the fluid collected inside the canister can cause the dissolvable material to dissolve when the canister is full. In some cases, the dissolvable material can start to dissolve when the fluid collected inside the canister reaches a level where the fluid covers at least a portion of one or both arms 520 of the fluid level sensor 518. Beneficially, the dissolvable material can slow the triggering of fluid level sensor and avoid triggering by transient splashes. Dissolution of the dissolvable material can cause the signal of the circuit created by the dissolvable material and the arms 520 to change and cause the fluid level sensor 518 to indicate a condition inside the canister (e.g., canister full condition). In some cases, the arms 520 can be covered with a semi-permeable membrane. The semi-permeable membrane can allow liquid particles in the fluid to move through the membrane by osmosis thereby causing the liquid to dissolve the dissolvable material. In some cases, the semi-permeable membrane can prevent any salts in the fluid from moving through the membrane thereby preventing the salts from dissolving the dissolvable material. The fluid level sensor 518 can include a warranty seal configuration in which the circuit formed by the arms 520 is normally closed. Damage to the fluid level sensor 518 can break the circuit (e.g., open the circuit) which can cause the fluid level sensor 518 to detect a condition inside the canister and/or detect damage to the circuit. In some cases, the fluid level sensor as disclosed in other examples herein can include a normally open circuit. Damage of a sensor in a normally open circuit can be hard to determine and can cause a failure of the sensor to measure a canister full condition. However, damage of and/or damage to the fluid level sensor 518 in a normally closed circuit can create a failure in the sensor which will case the fluid level sensor 518 to detect a canister full condition. Beneficially, in case of failure by the fluid level sensor 518, the fluid level sensor will detect a canister full condition thereby alerting the user and reducing the risk of harm and/or overflow.

In some cases, any of the fluid level sensors disclosed herein can include a material positioned between the arms 520 of the fluid level sensor 518, around the fluid level sensor 518, and/or any other position within the canister or adjacent to the fluid level sensor 518. The material can expand when fluid collected inside the canister contacts the material. In some cases, the material includes an expanding material such as gelling agent. In some cases, the expanding material can cause the arms 520 of the fluid level sensor 518 to move away from each other. This, in turn, can cause the signal of the fluid level sensor to change and indicate a condition inside the canister (e.g., canister full condition). In some cases, the expanding material can cause the circuit formed by the arms 520 to break through strain. This, in turn, can cause the signal of the fluid level sensor to change and indicate a condition inside the canister (e.g., canister full condition). In some cases, the arms 520 of the fluid level sensor 518, or any other components connected to the arms 520, can be pre-strained to promote deformation and break the circuit when the material expands. In some cases, the expanding material can be positioned around the arms 520. When the expanding materials expands, it can cause the arms 520 of the fluid level to move or spring toward each other so the arms 520 are closer to each other and/or in contact. This, in turn, can cause the signal of the fluid level sensor to change and indicate a condition inside the canister (e.g., canister full condition).

### Viscosity Measurements in the Canister

Measuring the viscosity of wound fluid within a fluid canister for a NPWT device is not currently done. Measuring the viscosity of the fluid can provide valuable information regarding wound healing and the condition of the wound. For example, low viscosity can indicate low protein content and high viscosity exudate can indicate high protein content, which may result from increased levels of bacteria in the wound or the inflammatory process. Measuring the viscosity of the fluid can be important as this may impact the treatment pathway a wound is put on, and can be used to monitor wound trajectory

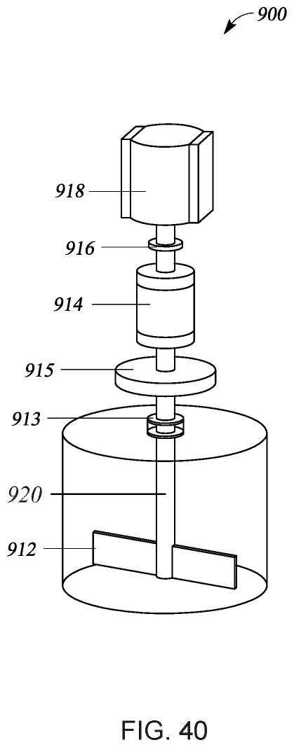

To measure the viscosity of the wound fluid within a canister, the principle of a rotational viscometer can be utilized. For example, the viscosity can be measured by calculating the torque output of a cylindrical shaped rotor which sits submerged within a substance in a sample chamber rotating at a constant speed. The rotational viscometer can be used to calculate the frictional force on the rotor caused by the test substance due to the level of extra energy required to maintain its spinning speed. FIG. 40 illustrates an example of a rotational viscometer 900 that can be used to provide a viscosity measurement of wound fluid within a canister. The rotational viscometer 900 can include a paddle 912, a bearing 913, a transducer 914, a torque limiter 915, a coupling 916, and a motor 918.

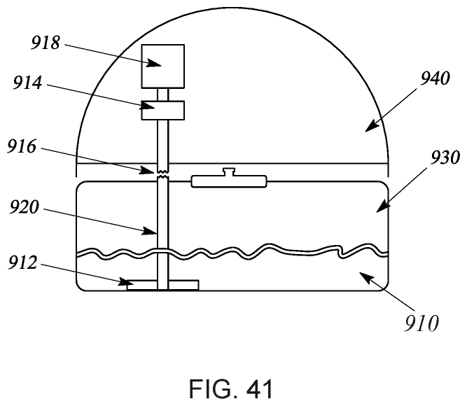

In some cases, the rotational viscometer 900 can include the paddle 912 and the axle 920 that can be manufactured within the canister component 930 of a negative pressure wound therapy device. The remaining circuitry (transducer 914, coupling 916, and motor 918) can be housed within the pump device 940 as shown in FIG. 41. When the canister 930 is attached to the pump device 940, the two parts of the construction come together, via a mechanical coupling 916, to complete the continuation of the axle and allow the motor 918 to spin the paddle 912, with the system measuring the required force and therefore viscosity of the fluid 910. In some cases, a test chamber is positioned within the interior of the canister. Additionally, or alternatively, the coupling 916 can include magnetic coupling. The test chamber can be used to store a portion of the fluid within the canister and the paddle is configured to be positioned in the test chamber.

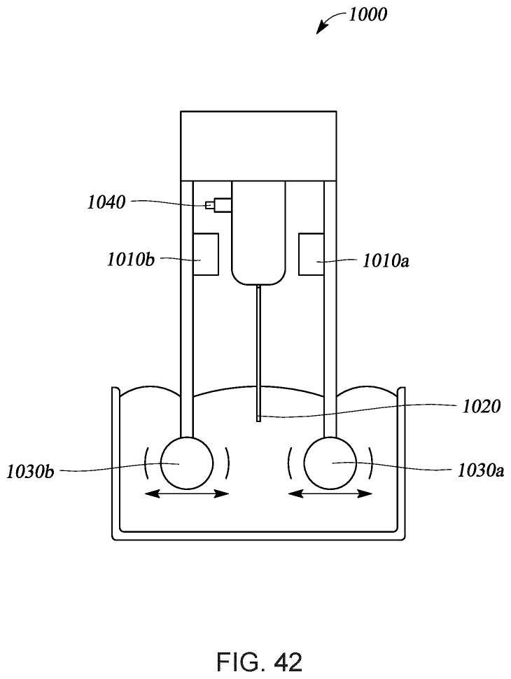

In some cases, to measure the viscosity of the wound fluid collected within the canister, the negative pressure therapy system can utilize a vibro-viscometer. The vibro-viscometer 1000 can work by sending uniform frequency vibrations out from two sensor plates through the fluid as illustrated in FIG. 42. A thicker fluid can require more driving current to maintain the vibrational frequency and therefore provide a measurement of the viscosity of the fluid. In some cases, the probes can be manufactured within the canister construction and the rest of the circuitry could be within the pump device 940 or housing of the pump.

The vibro-viscometer 1000 can include a one or more electromagnets 1010a, 1010b, a thermal sensor 1020, one or more vibrators 1030a, 1030b, and a displacement sensor 1040. Each of the electromagnets 1010a, 1010b can be mounted to the sensor plates, as shown in FIG. 42. In some cases, the thermal sensor 1020 can be in contact with the wound fluid collected within the canister and detect a temperature of the wound fluid. The vibrators 1030a, 1030b can be positioned on one end of the sensor plates so the vibrators 1030a, 1030b are in contact with the wound fluid collected within the canister. In some cases, the displacement sensor 1040 can detect and measure movement of the wound fluid collected inside the canister and provide an indication of the viscosity of the fluid.

In some cases, the device can utilize the negative pressure pump to pneumatically drive the motor 918 and/or the axle 920 of the vibro-viscometer 900. In some cases, the device can use different methods of mechanical coupling of the axle to allow the spinning of the paddle 912. In some cases, a test chamber is positioned within the interior of the canister. The test chamber can be used to store a portion of the fluid within the canister and the paddle 912 is configured to be positioned in the test chamber.

### Other Variations

Although some embodiments describe negative pressure wound therapy, the systems, devices, and/or methods disclosed herein can be applied to other types of therapies usable standalone or in addition to TNP therapy. Systems, devices, and/or methods disclosed herein can be extended to any medical device, and in particular any wound monitoring and/or treatment device. For example, systems, devices, and/or methods disclosed herein can be used with devices that provide one or more of ultrasound therapy, oxygen therapy, neurostimulation, microwave therapy, active agents, antibiotics, antimicrobials, or the like. Such devices can in addition provide TNP therapy. As another example, systems, devices, and/or methods disclosed herein can be used with a wound debridement system, patient monitoring system, or the like. The systems and methods disclosed herein are not limited to medical devices and can be utilized by any electronic device.

Any of transmission of data described herein can be performed securely. For example, one or more of encryption, https protocol, secure VPN connection, error checking, confirmation of delivery, or the like can be utilized.

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, can be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures or described herein may be implemented as software and/or firmware on a processor, controller, ASIC, FPGA, and/or dedicated hardware. The software or firmware can include instructions stored in a non-transitory computer-readable memory. The instructions can be executed by a processor, controller, ASIC, FPGA, or dedicated hardware. Hardware components, such as controllers, processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Conditional language used herein, such as, among others, “can,” “could”, “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language, such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A negative pressure wound therapy device comprising:
a device housing;
a negative pressure source supported by the device housing, the negative pressure source configured to provide negative pressure to a wound covered by a wound dressing;
a canister configured to be in fluid communication with the negative pressure source and the wound dressing, the canister comprising:
a canister housing configured to store fluid aspirated from the wound; and
a fluid level sensor supported by the canister, the fluid level sensor comprising a first arm and a second arm extending into an interior of the canister housing and configured to be in fluid communication with fluid aspirated from the wound; and
a material positioned between the first and second arms of the fluid level sensor, the material comprising an expanding material, the expanding material configured to expand when the fluid aspirated from the wound comes into contact with the expanding material causing the first and second arms to move away from each other;
wherein the first and second arms moving away from each other changes a signal of a circuit created by the first and second arms of the fluid level sensor; and
wherein the fluid level sensor is configured to detect a canister full condition based on the change of the signal of the circuit created by first and second arms of the fluid level sensor.

28. The negative pressure wound therapy device of claim 27, wherein the first and second arms moving away from each other causes the circuit created by the first and second arms to break thereby changing the signal of the circuit.

29. A negative pressure wound therapy device comprising:
a device housing;
a negative pressure source supported by the device housing, the negative pressure source configured to provide negative pressure to a wound covered by a wound dressing;
a canister configured to be in fluid communication with the negative pressure source and the wound dressing, the canister comprising:
a canister housing configured to store fluid aspirated from the wound; and
a fluid level sensor supported by the canister, the fluid level sensor comprising a first arm and a second arm extending into an interior of the canister housing and configured to be in fluid communication with fluid aspirated from the wound; and
a material positioned inside the canister, the material comprising an expanding material, the expanding material configured to expand when the fluid aspirated from the wound comes into contact with the expanding material causing a distance between the first and second arms to change;
wherein the change in distance between the first and second arms changes a signal of a circuit created by the first and second arms of the fluid level sensor; and
wherein the fluid level sensor is configured to detect a canister condition based on the change of the signal of the circuit created by first and second arms of the fluid level sensor.

30. The device of claim 29, wherein the expanding material causes the first and second arms to move toward each other, and wherein the first and second arms moving toward each other changes the signal of the circuit created by the first and second arms of the fluid level sensor.

31. A negative pressure wound therapy device comprising:
a device housing;
a negative pressure source supported by the device housing, the negative pressure source configured to provide negative pressure to a wound covered by a wound dressing;
a canister configured to be in fluid communication with the negative pressure source and the wound dressing, the canister comprising:
a canister housing configured to store fluid aspirated from the wound;
a cap connected to the canister housing and configured to be connected to the device housing when the canister is removably attached to the device housing; and
a fluid level sensor supported by the cap, the fluid level sensor comprising a first arm and a second arm extending into an interior of the canister housing and configured to be in fluid communication with fluid aspirated from the wound, wherein the fluid level sensor is configured to detect a level of fluid in the canister and/or a canister full condition responsive to the fluid aspirated from the wound coming into contact with the first and second arm of the fluid level sensor; and
an electronic circuitry configured to detect a state of the fluid level sensor and provide an indication of a status of the canister;
wherein the first and second arms comprise conductive, inductive, and/or capacitive path configured to facilitate detection of the level of fluid in the canister and/or a canister full condition.

32. The device of claim 31, wherein the first and second arms comprise a shape that changes along a length of the first and second arm.

33. The device of claim 31, wherein the first arm and the second arm have the same length.

34. The device of claim 31, wherein the first arm and the second arm are parallel to each other.

35. The device of claim 31, wherein the first arm and the second arm are angular to each other.

36. The device of claim 31, wherein the state of the fluid level sensor comprises a multi-bit value indicative of a level of fluid in the canister.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. The device of claim 27, wherein the first arm and the second arm comprise a shape that changes along a length of the first arm and the second arm.

63. The device of claim 27, wherein the first arm and the second arm have the same length.

64. The device of claim 27, wherein the first arm and the second arm are parallel to each other.

65. The device of claim 27, wherein the first arm and the second arm are angular to each other.

66. The device of claim 27, wherein the state of the fluid level sensor comprises a multi-bit value indicative of a level of fluid in the canister.

67. The device of claim 29, wherein the first arm and the second arm comprise a shape that changes along a length of the first arm and the second arm.

68. The device of claim 29, wherein the first arm and the second arm have the same length.

69. The device of claim 29, wherein the first arm and the second arm are parallel to each other.

70. The device of claim 29, wherein the first arm and the second arm are angular to each other.

71. The device of claim 29, wherein the state of the fluid level sensor comprises a multi-bit value indicative of a level of fluid in the canister.
